(19) United States
(12) Patent Application Publication
(10) Pub. No.: US 2004/0014748 A1
(43) Pub. Date: Jan. 22, 2004

(54) Use of microsomal triglyceride transfer protein (mtp) inhibitors for reducing the number of postprandiatriglyceride-rich lipoprotein particles (pptrl)

(76) Inventors: Rudi Grutzmann, Solingen (DE); Ulrich Muller, Wuppertal (DE); Hilmar Bischoff, Wuppertal (DE); Siegfried Zaiss, Wuppertal (DE)

Correspondence Address: JEFFREY M. GREENMAN, VICE PRESIDENT, PATENTS AND LICENSING, BAYER CORPORATION, 400 MORGAN LANE, WEST HAVEN, CT, 06516, US

(21) Appl. No.: 10/311,761

(22) PCT Filed: Jun. 8, 2001
(86) PCT No.: PCT/EP01/06526

(30) Foreign Application Priority Data
Jun. 21, 2000 (DE) 100-30-375.7

(51) Int. Cl. A61K 31/542; A61K 31/538; A61K 31/517; A61K 31/502; A61K 31/498; A61K 31/4745; A61K 31/4709

(52) U.S. Cl. 514/224.2; 514/300; 514/230.5; 514/248; 514/249; 514/266.21; 514/314

(57) ABSTRACT

The invention relates to the use of inhibitors of the microsomal triglyceride transfer protein (MTP) for reducing the number of postprandial triglyceride-rich lipoprotein particles (ppTRL) or for reducing their decomposition products i.e. the cholestcrol-rich “small remnant particle” (remnants). Said particles are associated with apolipoprotein B-48 (ApoB-48) and are designated as “ppTRL” in the further course of events.

The invention relates to the use of inhibitors of microsomal triglyceride transfer protein (MTP) for reducing the postprandial triglyceride-rich lipoprotein particles (ppTRL) and for reducing their degradation products, the cholesterol-richer small remnant particle (remnants). Said particles are associated with apolipoprotein B-48 (ApoB-48) and are referred to hereinafter as “ppTRL”.

Substances which inhibit the release of ApoB-100-associated lipoproteins are well known to the skilled worker. Such compounds are described for example in the publications EP 705 831, EP 779 279, EP 779 276, EP 802 198 and EP 799 828. These compounds, which reduce the plasma/serum levels of ApoB-100-associated lipoproteins, are MTP inhibitors. These applications mentioned also describe a rat test used to determine the effect of some substances on intestinal triglyceride absorption. In this rat test, the substances diminished the postprandial serum triglyceride increase.

Zaiss et al., Circulation 100 (18, Suppl. I):258 Abstr. 1343 (1999) describe the results of a mouse test. In this test, the MTP inhibitor implitapide prevents the formation of atherosclerotic plaques.

The prophylaxis and treatment of metabolic disorders, especially those affecting lipoprotein and lipid metabolism and associated with cardiovascular disorders and manifestations of neuronal degeneration, remains an essential aim of modern pharmaceutical research. In the literature there is discussion, for example, of alleles of the apoE genes which both represent risk factors for the development of coronary heart disease and are associated with the development of Alzheimer's disease (Rubinsztein, D. C. and Easton, D. F.; Apolipoprotein E genetic variation and Alzheimer's disease. a meta-analysis; Dement. Geriatr. Cogn. Disord., 1999; 10 (3): pp. 199-209; Nakayama S. and Kuzuhara S.; Apolipoprotein E phenotypes in healthy normal controls and demented subjects with Alzheimer's disease and vascular dementia in Mie Prefecture of Japan; Psychiatry Clin. Neurosci. 1999; 53 (6): pp. 643-648; Fullerton S. M., Strittmatter W. J. and Matthew W. D.; Peripheral sensory nerve defects in apolipoprotein E knockout mice; Exp. Neurol. 1998; 153 (1): pp. 156-163). ApoE is a constituent of the very low density lipoprotein (VLDL) which is produced in the liver, and of the chylomicrons which are synthesized in the intestine. ApoE mediates high-affinity binding of the chylomicrons and of VLDL to specific receptors on cells. This enables said particles to be metabolized and taken up into the corresponding cells, resulting in prevention of the accumulation of cholesterol-richer remnants and ppTRL in the plasma. Homozygous inactivation of the apoE genes, as is the case in apoE-knockout mice, results in apoE being undetectable in the serum of these animals. Development of the animals after birth is initially normal but they show disturbances of lipoprotein and lipid metabolism, which may be associated for example with plasma cholesterol levels which are elevated up to five-fold. In addition, these animals spontaneously develop manifestations of neuronal degeneration and atherosclerotic lesions. This is similar to the case of humans having an apoE variant which is able to bind only weakly or not at all to cellular receptors. However, ApoE is additionally involved in the regulation of the immune system, the regeneration of nerve cells and the differentiation of muscles (Masliah E, Mallory M, Ge N, Alford M, Veinbergs I, Roses A D Neurodegeneration in the central nervous system of apoE-deficient mice. Exp. Neurol. 1995; 136 (2): 107-122; Masliah E, Samuel W, Veinbergs I, Mallory M, Mante M, Saitoh T “Neurodegeneration and cognitive impairment in apoE-deficient mice is ameliorated by infusion of recombinant apoE” Brain Res 1997; 751(2):307-314; Chen Y, Lomnitski L, Michaelson D M, Shohami E “Motor and cognitive deficits in apolipoprotein E-deficient mice after closed head injury” Neuroscience 1997: 1255-1262; Fullerton S M, Strittmatter W J, Matthew W D. “Peripheral sensory nerve defects in apolipoprotein E knockout mice” Exp Neurol 1998; 153 (1): 156-63; Mato M, Ookawara S, Mashiko T, Sakamoto A, Mato T K, Maeda N, Kodama T Anat Rec 1999; 256 (2): 165-176 “Regional difference of lipid distribution in brain of apolipoprotein E deficient mice”).

The pathological consequences of disturbances of lipoprotein or lipid metabolism are accordingly not confined just to atherosclerosis. The apoE-knockout mouse is therefore suitable as animal model for investigating the effects of pharmaceuticals multifactorially on lipoprotein and lipid metabolism, atherosclerosis and damage to the nervous system with the aim of intervening in these multifaceted pathological processes.

In recent literature there are also descriptions of an important part played by, in particular, ppTRL and its degradation products in diabetes (Howard, B. V.; Insulin resistance and lipid metabolism; Am. J. Cardiol., 1999; 84 (1A): pp. 28J-32J; Mero, N., Malmstrom, R., Steiner, G., Taskinen, M., Syvanne, M.; Postprandial metabolism of apolipoprotein B-48- and B-100-containing particles in type 2 diabetes mellitus: relations to angiographically verified severity of coronary artery disease. Atherosclerosis, 2000; 150 (1): pp. 167-177). It is therefore of great importance to find possible ways of reducing the ppTRL levels in blood plasma.

It has now been found, surprisingly, that MTP inhibitors diminish ppTRL in the plasma, e.g. after lipid loading. The invention therefore relates to the use of MTP inhibitors for diminishing or reducing ppTRL in plasma. The lowering of the ppTRL by inhibition of MTP has a beneficial effect on morbidity and mortality, especially in relation to neurodegenerative and cardiovascular disorders. MTP inhibitors are therefore suitable for beneficially influencing these disease processes.

It has further been found, surprisingly, that the reduction in ppTRL, especially after intake of fatty food, occurs even with dosages of the MTP inhibitor with which no significant or only a slight reducing effect on the serum triglyceride or the serum cholesterol concentration is seen in the fasting state (about 12 hours after the last food intake). In the same way there is substantially no effect in this case on the LDL particles which originate from the liver and which, in humans, are exclusively ApoB-100-associated lipoprotein particles. A slight effect is intended to be regarded in this connection as a reduction in the plasma triglyceride or the plasma cholesterol concentration or a reduction in the ApoB-100-associated lipoproteins of less than 20%, preferably less than 10% or below. “Fasting plasma levels” means that measurements must not take place in postprandial plasma or serum, that is to say after intake of lipid-containing food, but in the fasting plasma or serum obtained about 12 hours after the last food intake.

Because of the effect on ppTRL, the MTP inhibitors can also be employed for inhibiting or diminishing intestinal cholesterol absorption.

Surprisingly, deliberate reduction in plasma ppTRL with a low dosage of an MTP inhibitor is itself sufficient to extend the survival of the patients, in conjunction with improved tolerability. Since disturbances of lipoprotein or lipid metabolism may, as explained above, lead to multifaceted degenerative disorders, the reduction in ppTRL makes an important therapeutic contribution to the treatment of such complex pathological states.

MTP inhibitors are described in the following documents, for example: Wetterau et al. Science 282, 751 (1998), J Lipid Res 37, 1468 (1996), Bristol-Myers-Squibb: EP-A-584 446, EP-A-643 057, WO 96/26205, WO 97/26240, WO 91/43255, WO 97/43257, WO 98/27979, U.S. Pat. No. 5,760,246, U.S. Pat. No. 5,827,875, WO 99/21564; Pfizer: WO 96/40 640, WO 98/23593, EP-A 887 345, WO 97/41111; Glaxo-Wellcome: WO 98/16526, WO 98/47877, WO 98/56790; Janssen: WO 96/13499, WO 96/33193; Novartis: WO 00/05201; Meji Seika Kaisha: WO 98/54135, Japan Tobacco: WO 99/31085; Advanced Medicine: WO 99/63929. In the following documents of Bayer A G, substances which inhibit the release of ApoB-100-associated lipoproteins are described, these substances being MTP inhbitors: EP-A 716 082, EP-A 719 763, EP-A 705 831, EP-A 753 517, EP-A 765 878, EP-A 764 647, EP-A 779 279, EP-A 779 276, EP-A 799 828, EP-A 802 198, EP-A 802 186, EP-A 802 188, EP-A 802 192, EP-A 802 197. The disclosure of the aforementioned documents disclosing MTP inhibitors is incorporated herein in its entirety by reference.

Some examples of MTP inhibitors described therein are listed below:

| Structure/systematic name (test number) | Described in |
|---|---|
| 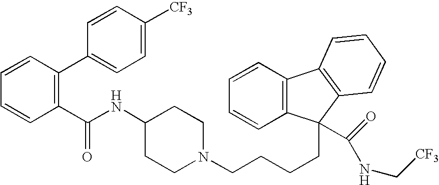 | EP-A 643 057, Wetterau et al., Science 282, 751 (1998) |
| N-(2,2,2-Trifluoroethyl)-9-{4-[4-({[4′-(trifluoromethyl)- | |
| 1,1′-biphenyl-2-yl]carbonyl}amino)-1-piperidinyl]butyl}- | |
| 9H-fluorene-9-carboxamide (BMS201038) | |
| 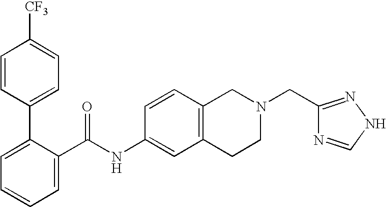 | WO 97/41111 (Pfizer) |
| N-[2-(1H-1,2,4-Triazol-3-ylmethyl)-1,2,3,4-tetrahydro-6- | |
| isoquinolinyl]-4′-(trifluoromethyl)-1,1′-biphenyl-2- | |
| carboxamide | |
| 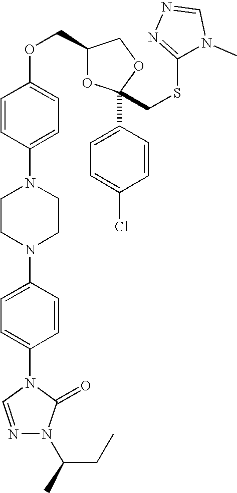 | WO 96/13499 (Janssen Int. NV) |
| 4-[4-(4-{4-[((2S,4S)-2-(4-Chlorophenyl)-2-{[(4-methyl- | |
| 4H-1,2,4-triazol-3-yl)sulfanyl]methyl}-1,3-dioxolan-4- | |
| yl)methoxy]phenyl}-1-piperazinyl)phenyl]-2-[(1R)-1- | |
| methylpropyl]-2,4-dihydro-3H-1,2,4-triazol-3-one | |
| (R103757) | |

Preferred MTP inhibitors which can be used according to the invention are: compounds of the general formula (A1)

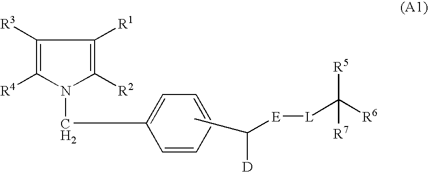

in which

R¹ and R² together form, with inclusion of the double bond connecting them, a phenyl or pyridyl ring or a ring of the formula

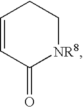

in which

R⁸ is hydrogen or straight-chain or branched alkyl having up to 4 carbon atoms,

R³ and R⁴ together form, with inclusion of the double bond connecting them, a phenyl ring or a 4- to 8-membered cycloalkene or oxocycloalkene residue, where all ring systems mentioned under R¹/R² and R³/R⁴ optionally have up to 3 identical or different halogen, trifluoromethyl, carboxyl, hydroxyl substituents, straight-chain or branched alkoxy or alkoxycarbonyl substituents each having up to 6 carbon atoms, or straight-chain or branched alkyl substituents which have up to 6 carbon atoms and which in turn may be substituted by hydroxyl or by straight-chain or branched alkoxy having up to 4 carbon atoms,

D is hydrogen, cycloalkyl having 4 to 12 carbon atoms or is straight-chain or branched alkyl having up to 12 carbon atoms,

E is the —CO— or —CS— group,

L is an oxygen or sulfur atom or is a group of the formula —NR⁹,

in which

R⁹ is hydrogen or straight-chain or branched alkyl which has up to 6 carbon atoms and which is optionally substituted by hydroxyl or phenyl,

R⁵ is phenyl or is a 5- to 7-membered saturated or unsaturated heterocycle having up to 3 heteroatoms from the series S, N and/or O, where the rings optionally have up to 3 identical or different nitro, carboxyl, halogen, cyano substituents or straight-chain or branched alkenyl or alkoxycarbonyl substituents each having up to 6 carbon atoms or straight-chain or branched alkyl substituents which have up to 6 carbon atoms and which are optionally substituted by hydroxyl, carboxyl or by straight-chain or branched alkoxy or alkoxycarbonyl each having up to 6 carbon atoms, and/or the rings are optionally substituted by a group of the formula —OR¹⁰ or —NR¹¹R¹²,

in which

R¹⁰ is hydrogen or straight-chain or branched alkyl or alkenyl each having up to 6 carbon atoms,

R¹¹ and R¹² are identical or different and are phenyl, hydrogen or straight-chain or branched alkyl having up to 6 carbon atoms, or straight-chain or branched acyl which has up to 8 carbon atoms and which is optionally substituted by a group of the formula —NR¹³R¹⁴,

in which

R¹³ and R¹⁴ are identical or different and are hydrogen or straight-chain or branched acyl having up to 8 carbon atoms,

R⁶ is hydrogen, carboxyl or is straight-chain or branched alkoxycarbonyl having up to 5 carbon atoms, or is straight-chain or branched alkyl which has up to 6 carbon atoms and which is optionally substituted by hydroxyl or by a group of the formula —O—CO—R¹⁵,

in which

R¹⁵ is phenyl which optionally has up to 3 identical or different halogen, hydroxyl substituents or straight-chain or branched alkyl substituents having up to 5 carbon atoms, or straight-chain or branched alkyl or alkenyl which each have up to 22 carbon atoms and which are optionally substituted by a group of the formula —OR¹⁶,

in which

R¹⁶ is hydrogen, benzyl, triphenylmethyl or straight-chain or branched acyl having up to 6 carbon atoms,

R⁷ is hydrogen or

R⁶ and R⁷ together are the group of the formula ═O,

or of the general formula (A2)

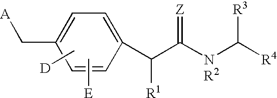

in which

A is a radical of the formula

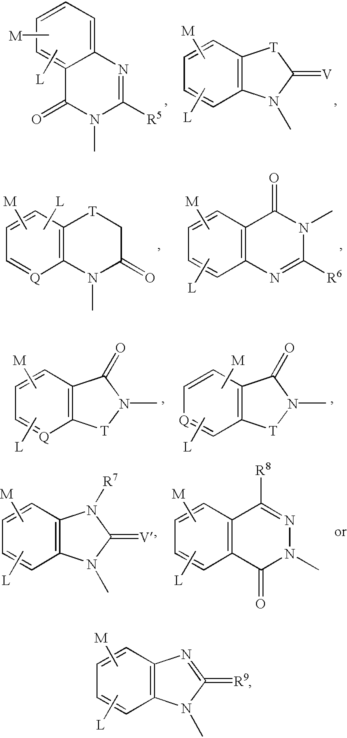

in which

L and M are identical or different and are hydrogen, halogen, trifluoromethyl, carboxyl, cycloalkyl having 3 to 6 carbon atoms, hydroxyl, phenyl or straight-chain or branched alkyl, alkoxycarbonyl or alkoxy each having up to 6 carbon atoms,

Q is a nitrogen atom or the —CH group,

T is a group of the formula —SO₂ or —CO or an oxygen or sulfur atom,

V is an oxygen or sulfur atom,

R⁵, R⁶, R⁷ and R⁸ are identical or different and are hydrogen, straight-chain or branched alkyl having up to 6 carbon atoms, benzyl or phenyl, which are optionally substituted by halogen or by straight-chain or branched alkyl having up to 6 carbon atoms,

R⁹ is trifluoromethyl, benzyl or a 5- to 7-membered, optionally benzo-fused heterocycle having up to 3 heteroatoms from the series S, N and/or O and optionally having up to 3 identical or different halogen, phenyl, hydroxyl substituents or straight-chain or branched alkyl or alkoxy substituents each having up to 4 carbon atoms, or is a group of the formula —S(O)ₐ—R¹⁰,

in which

a is a number 0, 1 or 2,

R¹⁰ is straight-chain or branched alkyl or alkenyl which each have up to 8 carbon atoms and which are optionally substituted by straight-chain or branched acyl having up to 6 carbon atoms or by aryl or aroyl each of which have up to 10 carbon atoms and which may in turn have up to 2 identical or different halogen, trifluoromethyl substituents or straight-chain or branched acyl substituents having up to 5 carbon atoms, or is aryl which has 6 to 10 carbon atoms and which is optionally substituted by halogen, hydroxyl, trifluoromethyl or straight-chain or branched alkyl or alkoxy each having up to 5 carbon atoms,

D and E are identical or different and are hydrogen, halogen, trifluoromethyl, hydroxyl, carboxyl or straight-chain or branched alkyl, alkoxy or alkoxycarbonyl each having up to 6 carbon atoms,

Z is an oxygen or sulfur atom,

R¹ is cycloalkyl having 3 to 10 carbon atoms or is straight-chain or branched alkyl having 1 to 10 carbon atoms, or is phenyl which optionally has up to 2 identical or different halogen, nitro, cyano, hydroxyl substituents or straight-chain or branched alkyl or alkoxy substituents each having up to 4 carbon atoms,

R² is hydrogen or straight-chain or branched alkyl having up to 3 carbon atoms,

R³ is hydrogen or straight-chain or branched alkyl having up to 5 carbon atoms, or is is cycloalkyl having 3 to 7 carbon atoms, or is phenyl or a 5- to 7-membered aromatic heterocycle having up to 3 heteroatoms from the series S, N and/or O, which optionally have up to 3 identical or different halogen, nitro, phenyl, hydroxyl substituents or straight-chain or branched alkyl or alkoxy substituents having up to 6 carbon atoms,

R⁴ is hydrogen or is a group of the formula —CH₂—OH or CH₂O—CO—R¹¹,

in which

R¹¹ is hydrogen, straight-chain or branched alkyl having up to 8 carbon atoms or phenyl which optionally has up to 3 identical or different halogen, hydroxyl, cyano substituents or straight-chain or branched alkyl or alkoxy substituents each having up to 4 carbon atoms,

or of the general formula (A3)

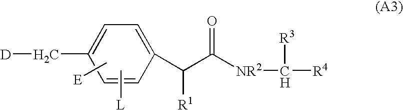

in which

D is a radical of the formula

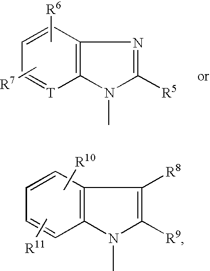

T is a nitrogen atom or the —CH group,

R⁶, R⁷, R¹⁰ and R¹¹ are identical or different and are hydrogen, trifluoromethyl, halogen or straight-chain or branched alkyl or alkoxy each having up to 6 carbon atoms,

R⁵, R⁸ and R⁹ are identical or different and are hydrogen, cycloalkyl having 3 to 6 carbon atoms, phenyl, straight-chain or branched alkoxycarbonyl having up to 6 carbon atoms or straight-chain or branched alkyl which has up to 6 carbon atoms and which is optionally substituted by halogen, or, in the case where T is a nitrogen atom, R⁵ can also be benzyl,

E and L are identical or different and are hydrogen, halogen, trifluoromethyl, hydroxyl, carboxyl or straight-chain or branched alkyl, alkoxy or alkoxycarbonyl each having up to 6 carbon atoms,

R¹ is cycloalkyl having 3 to 10 carbon atoms or is straight-chain or branched alkyl having 1 to 10 carbon atoms, or is phenyl which optionally has up to 2 identical or different halogen, cyano, hydroxyl substituents or straight-chain or branched alkyl or alkoxy substituents each having up to 4 carbon atoms,

R² is hydrogen or straight-chain or branched alkyl having up to 3 carbon atoms,

R³ is hydrogen or straight-chain or branched alkyl having up to 5 carbon atoms, or is cycloalkyl having 3 to 7 carbon atoms, or is phenyl or a 5- to 7-membered aromatic heterocycle having up to 3 heteroatoms from the series S, N and/or O, which optionally have up to 3 identical or different halogen, nitro, phenyl, hydroxyl substituents or straight-chain or branched alkyl or alkoxy substituents having up to 6 carbon atoms,

R⁴ is hydrogen or is a group of the formula —CH₂—OH or CH₂O—CO—R¹²,

in which

R¹² is hydrogen, straight-chain or branched alkyl having up to 8 carbon atoms or phenyl which optionally has up to 3 identical or different halogen, hydroxyl, cyano substituents or straight-chain or branched alkyl or alkoxy substituents each having up to 4 carbon atoms,

or of the general formula (A4)

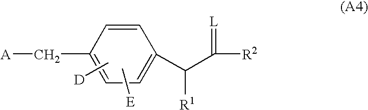

in which

A is a radical of the formula

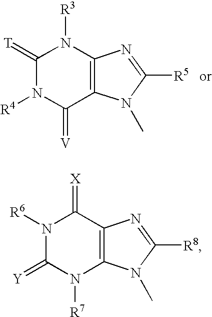

in which

R³, R⁴, R⁶ and R⁷ are identical or different and are hydrogen, cycloalkyl having 3 to 7 carbon atoms or aryl having 6 to 10 carbon atoms, or straight-chain or branched alkyl or alkenyl which each have up to 8 carbon atoms and which are optionally substituted by halogen, hydroxyl or aryl having 6 to 10 carbon atoms,

T, V, X and Y are identical or different and are an oxygen or sulfur atom,

R⁵ and R⁸ are identical or different and are hydrogen, halogen, cycloalkyl having 3 to 8 carbon atoms or straight-chain or branched alkyl or alkenyl which each have up to 8 carbon atoms and which are optionally substituted by cycloalkyl having 3 to 8 carbon atoms or by a 5- to 6-membered aromatic, optionally benzo-fused heterocycle having up to 3 heteroatoms from the series S, N and/or O, or by aryl having 6 to 10 carbon atoms, where the rings in turn may have up to 3 identical or different 5- to 6-membered aromatic heterocyclic substituents having up to 3 heteroatoms from the series S, N and/or O, or phenyl, benzyl, halogen, hydroxyl, carboxyl substituents or straight-chain or branched alkyl, alkoxy or alkoxycarbonyl substituents each having up to 6 carbon atoms, or aryl having 6 to 10 carbon atoms or a 5- to 7-membered aromatic, optionally benzo-fused heterocycle having up to 3 heteroatoms from the series S, N and/or O, which optionally have up to 3 identical or different halogen, phenyl, trifluoromethyl, hydroxyl, carboxyl substituents or straight-chain or branched alkyl, alkoxy or alkoxycarbonyl substituents each having up to 6 carbon atoms, or a substituent group of the formula —(CO)ₐ—NR⁹R¹⁰,

in which

a is a number 0 or 1,

R⁹ and R¹⁰ are identical or different and are hydrogen, phenyl or straight-chain or branched alkyl or acyl each having up to 5 carbon atoms,

D and E are identical or different and are hydrogen, halogen, trifluoromethyl, hydroxyl, carboxyl or are straight-chain or branched alkyl, alkoxy or alkoxycarbonyl each having up to 6 carbon atoms,

R¹ is hydrogen or cycloalkyl having 3 to 8 carbon atoms, or is straight-chain or branched alkyl or alkenyl which each have up to 8 carbon atoms and which are optionally substituted by cycloalkyl having 3 to 6 carbon atoms, phenyl or by a 5- to 6-membered aromatic heterocycle having up to 3 heteroatoms from the series S, N and/or O, or is phenyl or a 5- to 6-membered aromatic heterocycle having up to 3 heteroatoms from the series S, N and/or O, where the ring systems optionally have up to 3 identical or different halogen, phenyl, trifluoromethyl substituents or straight-chain or branched alkyl or alkoxy substituents each having up to 5 carbon atoms, hydroxyl substituents or a substituent group of the formula —NR¹¹R¹²,

in which

R¹¹ and R¹² have the abovementioned meaning of R⁹ and R¹⁰ and are identical to or different from the latter,

L is an oxygen or sulfur atom,

R² is mercapto, hydroxyl, straight-chain or branched alkoxy having up to 8 carbon atoms or the group of the formula

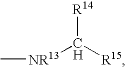

in which

R¹³ is hydrogen or straight-chain or branched alkyl having up to 4 carbon atoms,

R¹⁴ is hydrogen, phenyl or a 5- to 6-membered aromatic heterocycle having up to 3 heteroatoms from the series S, N and/or O,

R¹⁵ is hydrogen or straight-chain or branched alkyl which has up to 8 carbon atoms and is optionally substituted by hydroxyl,

or of the general formula (A5)

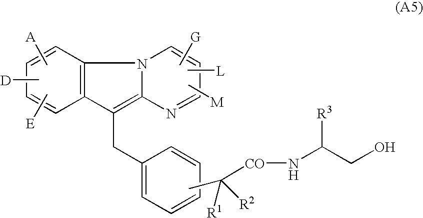

in which

A, D, E, G, L and M are identical or different and are hydrogen, halogen, trifluoromethyl, carboxyl, hydroxyl, straight-chain or branched alkoxy or alkoxycarbonyl each having up to 6 carbon atoms or straight-chain or branched alkyl which has up to 6 carbon atoms and which in turn may be substituted by hydroxyl or by straight-chain or branched alkoxy having up to 4 carbon atoms,

R¹ and R² are identical or different and are hydrogen, cycloalkyl having 3 to 8 carbon atoms or straight-chain or branched alkyl which has up to 10 carbon atoms and which is optionally substituted by cycloalkyl having 3 to 6 carbon atoms, or are phenyl which is optionally substituted by halogen or trifluoromethyl, or

R¹ and R² form, together with the carbon atom, a 4-8-membered cycloalkyl ring, and

R³ is phenyl which optionally has up to 3 identical or different nitro, carboxyl, halogen, cyano substituents or straight-chain or branched alkenyl or alkoxycarbonyl substituents each having up to 6 carbon atoms, or straight-chain or branched alkyl substituents which have up to 6 carbon atoms and which are optionally substituted by hydroxyl, carboxyl or by straight-chain or branched alkoxy or alkoxycarbonyl each having up to 6 carbon atoms, and/or is optionally substituted by a group of the formula —OR⁴ or —NR⁵R⁶,

in which

R⁴ is hydrogen or straight-chain or branched alkyl or alkenyl each having up to 6 carbon atoms,

R⁵ and R⁶ are identical or different and are phenyl, hydrogen or straight-chain or branched alkyl having up to 6 carbon atoms, or straight-chain or branched acyl which has up to 8 carbon atoms and which is optionally substituted by a group of the formula —NR⁷R⁸,

in which

R⁷ and R⁸ are identical or different and are hydrogen or straight-chain or branched acyl having up to 8 carbon atoms,

or of the general formula (A6)

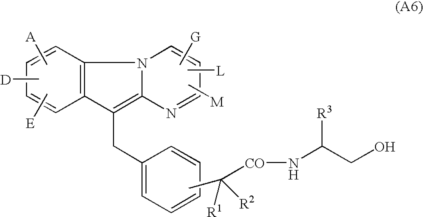

in which

A, D, E, G, L and M are identical or different and are hydrogen, halogen, trifluoromethyl, carboxyl, hydroxyl, straight-chain or branched alkoxy or alkoxycarbonyl each having up to 6 carbon atoms or straight-chain or branched alkyl which has up to 6 carbon atoms and which in turn may be substituted by hydroxyl or by straight-chain or branched alkoxy having up to 4 carbon atoms,

R¹ and R² are identical or different and are hydrogen, cycloalkyl having 3 to 8 carbon atoms or straight-chain or branched alkyl which has up to 10 carbon atoms and which is optionally substituted by cycloalkyl having 3 to 6 carbon atoms, or are phenyl which is optionally substituted by halogen or trifluoromethyl, or

R¹ and R² form, together with the carbon atom, a 4-8-membered cycloalkyl ring,

and

R³ is phenyl which optionally has up to 3 identical or different nitro, carboxyl, halogen, cyano substituents or straight-chain or branched alkenyl or alkoxycarbonyl substituents each having up to 6 carbon atoms, or straight-chain or branched alkyl substituents which have up to 6 carbon atoms and which are optionally substituted by hydroxyl, carboxyl or by straight-chain or branched alkoxy or alkoxycarbonyl each having up to 6 carbon atoms, and/or is optionally substituted by a group of the formula —OR⁴ or —NR⁵R⁶,

in which

R⁴ is hydrogen or straight-chain or branched alkyl or alkenyl each having up to 6 carbon atoms,

R⁵ and R⁶ are identical or different and are phenyl, hydrogen or straight-chain or branched alkyl having up to 6 carbon atoms, or are straight-chain or branched acyl which has up to 8 carbon atoms and which is optionally substituted by a group of the formula —NR⁷R⁸,

in which

R⁷ and R⁸ are identical or different and are hydrogen or straight-chain or branched acyl having up to 8 carbon atoms,

where appropriate in an isomeric form and the salts thereof.

MTP inhibitors of great interest are the compounds of the general formula (A1), and likewise of particular importance are the compounds of the following Examples 1 to 119, in particular the compounds of Examples 92 to 119, very particularly the compounds of Examples 48 and 80, (2S)-2-cyclopentyl-2-[4-(2,4-dimethyl-pyrido[2,3-b]indol-9-ylmethyl)-phenyl]-N-(2-(1R)-hydroxy-1-phenyl-ethyl)-acetamide (Example 48) and (2S)-2-cyclopentyl-2-[4-(2,4-dimethyl-pyrimido[1,2-a]indol-10-ylmethyl)-phenyl]-N-(2-(1R)-hydroxy-1-phenyl-ethyl)-acetamide (Example 80).

The use of the physiologically acceptable salts of the MTP inhibitors mentioned above is also claimed within the scope of the present invention. Physiologically acceptable salts of the compounds of the invention are, for example, salts of the substances of the invention with mineral acids, carboxylic acids or sulfonic acids. Particularly preferred examples are salts with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid.

Physiologically acceptable salts of the MTP inhibitors mentioned above may likewise be metal or ammonium salts of the compounds of the invention having a free carboxyl group. Particularly preferred examples are sodium, potassium, magnesium or calcium salts, and ammonium salts derived from ammonia or organic amines such as, for example, ethylamine, di- or triethylamine, ethanolamine, di- or triethanolamine, dicyclohexylamine, dimethylaminoethanol, arginine, lysine, ethylenediamine or 2-phenylethylamine.

The MTP inhibitors of the invention may exist in stereoisomeric forms which either are related as image and mirror image (enantiomers) or are not related as image and mirror image (diastereomers). The invention relates both to the enantiomers and diastereomers or respective mixtures thereof. These mixtures of enantiomers and diastereomers can be separated into the stereoisomerically pure constituents in a known manner.

Because of their effect in particular on the ppTRL plasma levels, the MTP inhibitors can be employed for the prophylaxis and treatment of disorders associated with elevated plasma levels of ppTRL and their remnants. Those which may be mentioned are: disorders of the cardiovascular system such as, for example, atherosclerosis or myocardial infarction, also those disorders which can be attributed to manifestations of neuronal degeneration, and are associated with impairments of the metabolism of lipoproteins or lipids, e.g. dementia or Alzheimer's disease. Impairments of carbohydrate metabolism such as, for example, diabetes or IGT (impaired glucose tolerance) are likewise associated with elevated and more persistent ppTRL levels. These disorders can therefore also be treated with MTP inhibitors.

The reduction of ppTRL also brings about a diminished formation of their degradation products, the remnants. Since the ppTRL and the remnants are associated with ApoB-48, the MTP inhibitors bring about not only a reduction of ppTRL but also a reduction of the remnants and of ApoB-48 and ApoB-48-associated lipoproteins.

Preferred MTP inhibitors are the compounds listed in the following table:

| Ex. | | |
|---|---|---|
| No. | Structure | Name |
| 1 | 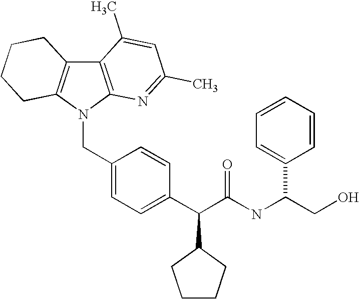 | 2-Cyclopentyl-2-[4-(2,4-dimethyl- 5,6,7,8-tetrahydro-pyrido[2,3- b]indol-9-ylmethyl)-phenyl]-N-(2- hydroxy-1-phenyl-ethyl)- acetamide |
| 2 | 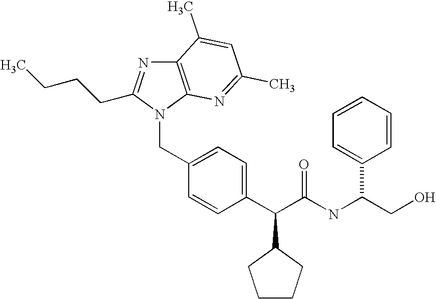 | 2-[4-(2-Butyl-benzoimidazol-1- ylmethyl)-phenyl]-2-cyclopentyl- N-(2-hydroxy-1-phenyl-ethyl)-acetamide |
| 3 | 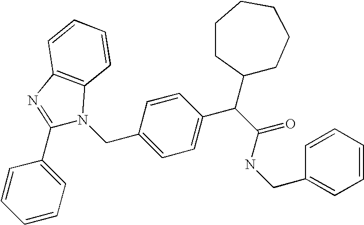 | N-Benzyl-2-cycloheptyl-2-[4-(2- phenyl-benzoimidazol-1-ylmethyl)-phenyl]-acetamide |
| 4 | 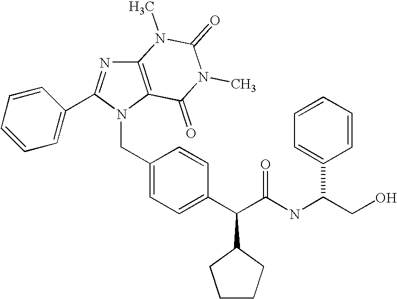 | 2-Cyclopentyl-2-[4-(1,3-dimethyl- 2,6-dioxo-8-phenyl-1,2,3,6- tetrahydro-purin-7-ylmethyl)- phenyl]-N-(2-hydroxy-1-phenyl-ethyl)-acetamide |
| 5 | 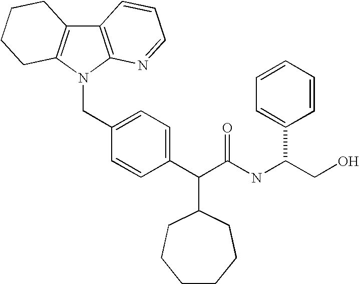 | 2-Cycloheptyl-N-(2-hydroxy-1- phenyl-ethyl)-2-[4-(5,6,7,8- tetrahydro-pyrido[2,3-b]indol-9- ylmethyl)-phenyl]-acetamide |
| 6 | 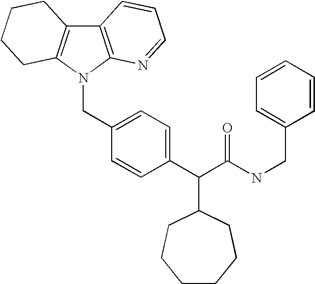 | N-Benzyl-2-cycloheptyl-2-[4- (5,6,7,8-tetrahydro-pyrido[2,3- b]indol-9-ylmethyl)-phenyl]- acetamide |
| 7 | 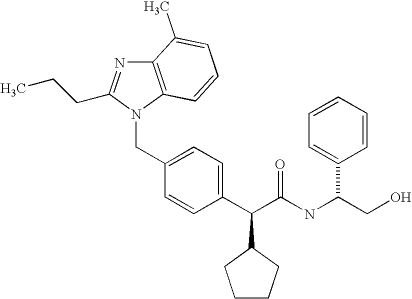 | 2-Cyclopentyl-N-(2-hydroxy-1- phenyl-ethyl)-2-[4-(4-methyl-2- propyl-benzoimidazol-1- ylmethyl)-phenyl]-acetamide |
| 8 | 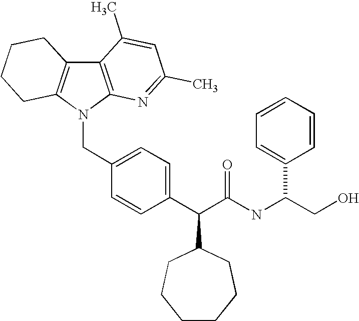 | 2-Cycloheptyl-2-[4-(2,4-dimethyl- 5,6,7,8-tetrahydro-pyrido[2,3- b]indol-9-ylmethyl)-phenyl]-N-(2- hydroxy-1-phenyl-ethyl)- acetamide |
| 9 | 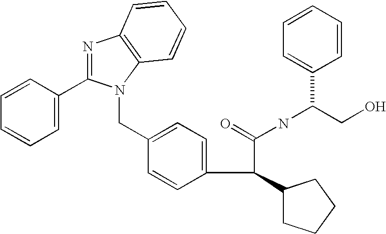 | 2-Cyclopentyl-N-(2-hydroxy-1- phenyl-ethyl)-2-[4-(2-phenyl- benzoimidazol-1-ylmethyl)- phenyl]-acetamide |
| 10 | 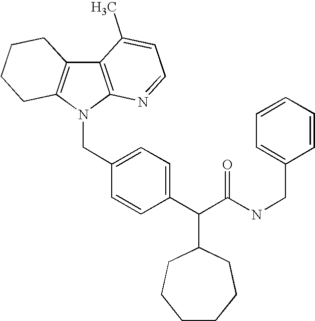 | N-Benzyl-2-cycloheptyl-2-[4-(4- methyl-5,6,7,8-tetrahydro- pyrido[2,3-b]indol-9-ylmethyl)- phenyl]-acetamide |
| 11 | 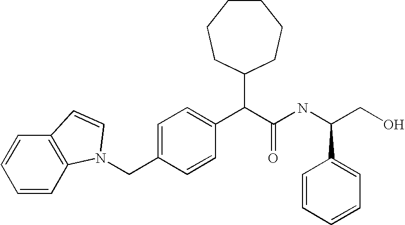 | 2-Cycloheptyl-N-(2-hydroxy-1- phenyl-ethyl)-2-(4-indol-1- ylmethyl-phenyl)-acetamide |
| 12 | 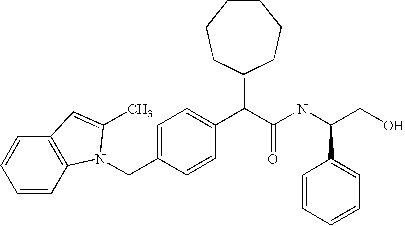 | 2-Cycloheptyl-N-(2-hydroxy-1- phenyl-ethyl)-2-[4-(2-methyl- indol-1-ylmethyl)-phenyl]-acetamide |
| 13 | 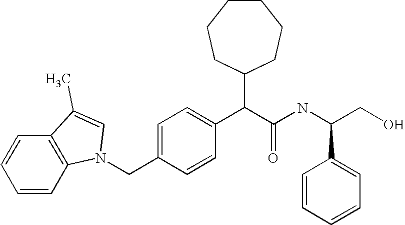 | 2-Cycloheptyl-N-(2-hydroxy-1- phenyl-ethyl)-2-[4-(3-methyl- indol-1-ylmethyl)-phenyl]- acetamide |
| 14 | 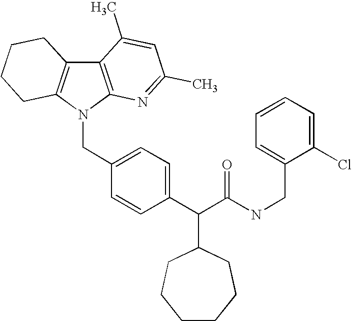 | N-(2-Chloro-benzyl)-2- cycloheptyl-2-[4-(2,4-dimethyl- 5,6,7,8-tetrahydro-pyrido[2,3- b]indol-9-ylmethyl)-phenyl]- acetamide |
| 15 | 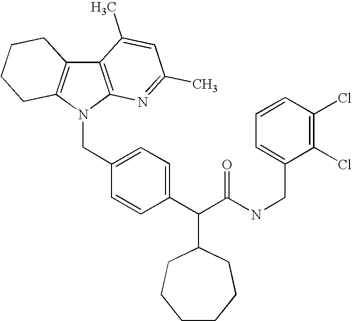 | N-(3-Chloro-benzyl)-2- cycloheptyl-2-[4-(2,4-dimethyl- 5,6,7,8-tetrahydro-pyrido[2,3- b]indol-9-ylmethyl)-phenyl]- acetamide |
| 16 | 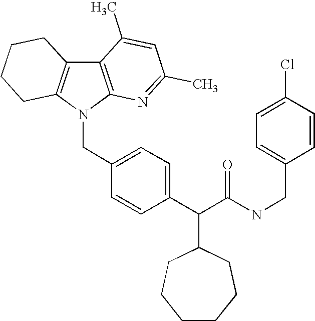 | N-(4-Chloro-benzyl)-2- cycloheptyl-2-[4-(2,4-dimethyl- 5,6,7,8-tetrahydro-pyrido[2,3- b]indol-9-ylmethyl)-phenyl]- acetamide |
| 17 | 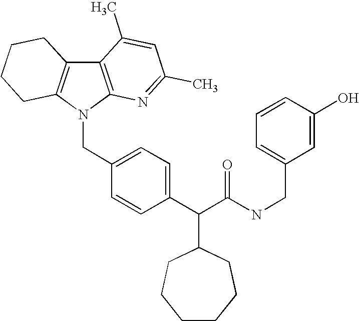 | 2-Cycloheptyl-2-[4-(2,4-dimethyl- 5,6,7,8-tetrahydro-pyrido[2,3- b]indol-9-ylmethyl)-phenyl]-N-(3- hydroxy-benzyl)-acetamide |
| 18 | 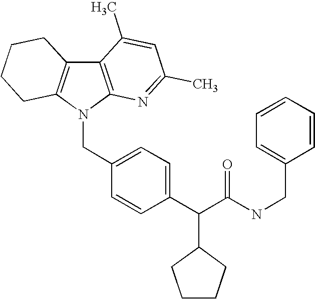 | N-Benzyl-2-cyclopentyl-2-[4-(2,4- dimethyl-5,6,7,8-tetrahydro- pyrido[2,3-b]indol-9-ylmethyl)- phenyl]-acetamide |
| 19 | 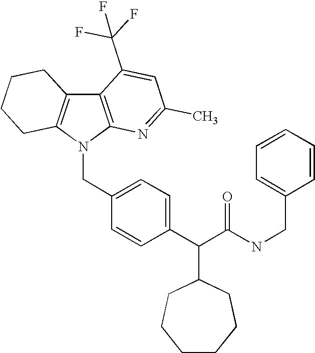 | N-Benzyl-2-cycloheptyl-2-[4-(2- methyl-4-trifluoromethyl-5,6,7,8- tetrahydro-pyrido[2,3-b]indol-9- ylmethyl)-phenyl]-acetamide |
| 20 | 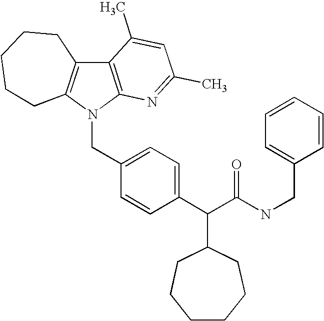 | N-Benzyl-2-cycloheptyl-2-[4-(2,4- dimethyl-6,7,8,9-tetrahydro-5H- 1,10-diaza-benzo[a]azulen-10- ylmethyl)-phenyl]-acetamide |
| 21 | 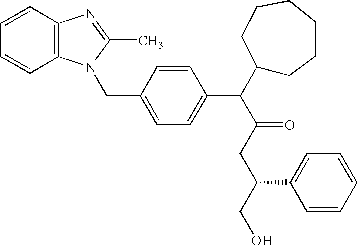 | 2-Cycloheptyl-N-(2-hydroxy-1- phenyl-ethyl)-2-[4-(2-methyl- benzoimidazol-1-ylmethyl)- phenyl]-acetamide |
| 22 | 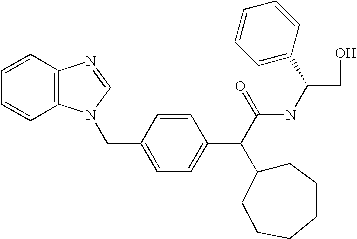 | 2-(4-Benzoimidazol-1-ylmethyl- phenyl)-2-cycloheptyl-N-(2- hydroxy-1-phenyl-ethyl)- acetamide |
| 23 | 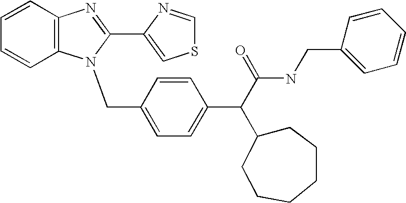 | N-Benzyl-2-cycloheptyl-2-[4-(2- thiazol-4-yl-benzoimidazol-1- ylmethyl)-phenyl]-acetamide |
| 24 | 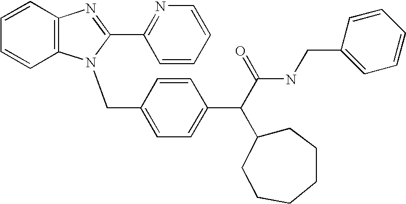 | N-Benzyl-2-cycloheptyl-2-[4-(2- pyridin-2-yl-benzoimidazol-1- ylmethyl)-phenyl]-acetamide |
| 25 | 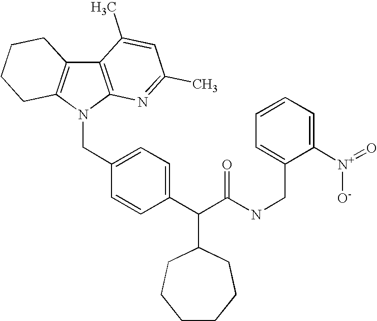 | 2-Cycloheptyl-2-[4-(2,4-dimethyl- 5,6,7,8-tetrahydro-pyrido[2,3- b]indol-9-ylmethyl)-phenyl]-N-(2- nitro-benzyl)-acetamide |
| 26 | 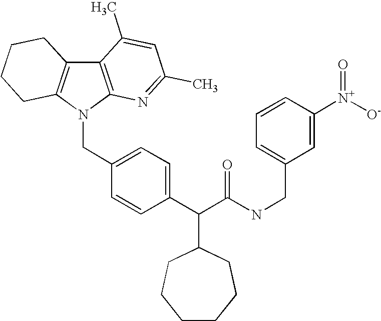 | 2-Cycloheptyl-2-[4-(2,4-dimethyl- 5,6,7,8-tetrahydro-pyrido[2,3- b]indol-9-ylmethyl)-phenyl]-N-(3- nitro-benzyl)-acetamide |
| 27 | 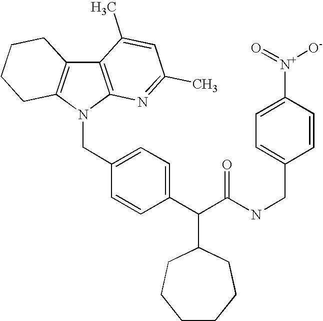 | 2-Cycloheptyl-2-[4-(2,4-dimethyl- 5,6,7,8-tetrahydro-pyrido[2,3-b]indol-9-ylmethyl)-phenyl]-N-(4- nitro-benzyl)-acetamide |
| 28 | 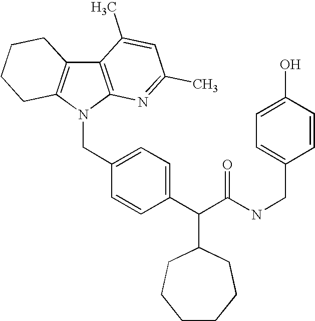 | 2-Cycloheptyl-2-[4-(2,4-dimethyl- 5,6,7,8-tetrahydro-pyrido[2,3-b]indol-9-ylmethyl)-phenyl]-N-(4- hydroxy-benzyl)-acetamide |
| 29 | 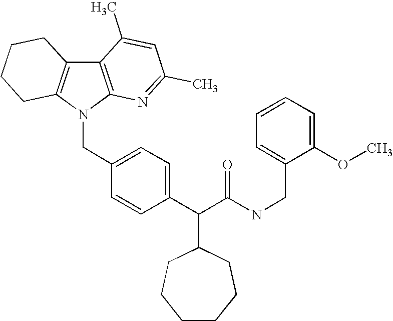 | 2-Cycloheptyl-2-[4-(2,4-dimethyl- 5,6,7,8-tetrahydro-pyrido[2,3- b]indol-9-ylmethyl)-phenyl]-N-(2- methoxy-benzyl)-acetamide |
| 30 | 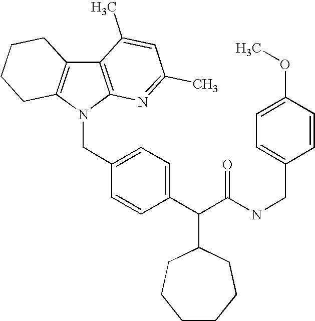 | 2-Cycloheptyl-2-[4-(2,4-dimethyl- 5,6,7,8-tetrahydro-pyrido[2,3- b]indol-9-ylmethyl)-phenyl]-N-(4- methoxy-benzyl)-acetamide |
| 31 | 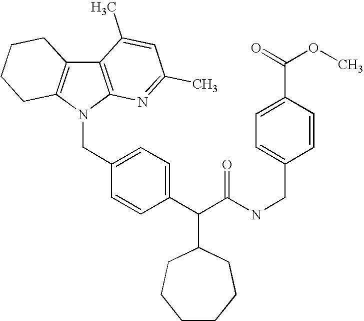 | 4-({2-Cycloheptyl-2-[4-(2,4- dimethyl-5,6,7,8-tetrahydro- pyrido[2,3-b]indol-9-ylmethyl)- phenyl]-acetylamino}-methyl)- benzoic acid methyl ester |
| 32 | 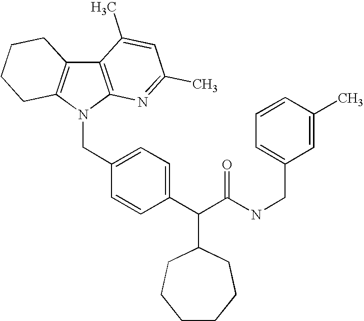 | 2-Cycloheptyl-2-[4-(2,4-dimethyl- 5,6,7,8-tetrahydro-pyrido[2,3- b]indol-9-ylmethyl)-phenyl]-N-(3- methyl-benzyl)-acetamide |
| 33 | 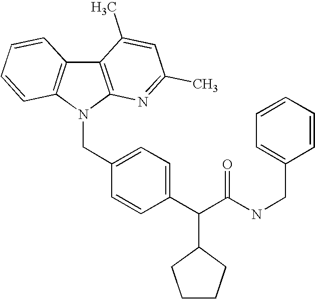 | N-Benzyl-2-cyclopentyl-2-[4-(2,4- dimethyl-pyrido[2,3-b]indol-9- ylmethyl)-phenyl]-acetamide |
| 34 | 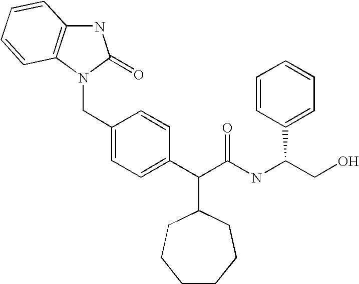 | 2-Cycloheptyl-N-(2-hydroxy-1- phenyl-ethyl)-2-[4-(2-thioxo-2,3- dihydro-benzoimidazol-1- ylmethyl)-phenyl]-acetamide |
| 35 | 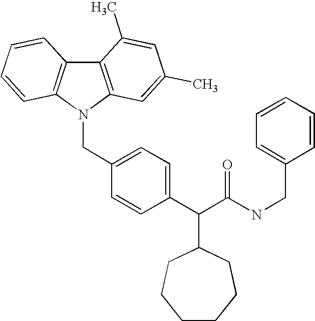 | N-Benzyl-2-cycloheptyl-2-[4-(2,4- dimethyl-pyrido[2,3-b]indol-9- ylmethyl)-phenyl]-acetamide |
| 36 | 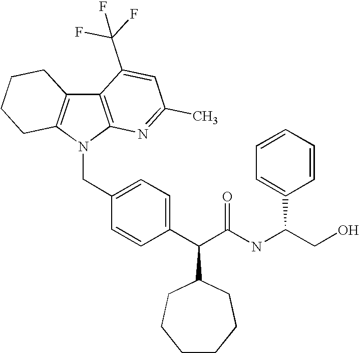 | 2-Cycloheptyl-N-(2-hydroxy-1- phenyl-ethyl)-2-[4-(2-methyl-4- trifluoromethyl-5,6,7,8-tetrahydro- pyrido[2,3-b]indol-9-ylmethyl)- phenyl]-acetamide |
| 37 | 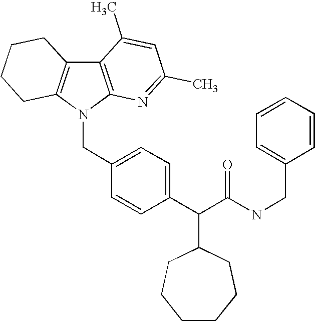 | N-Benzyl-2-cycloheptyl-2-[4-(2,4- dimethyl-5,6,7,8-tetrahydro- pyrido[2,3-b]indol-9-ylmethyl)- phenyl]-acetamide |
| 38 | 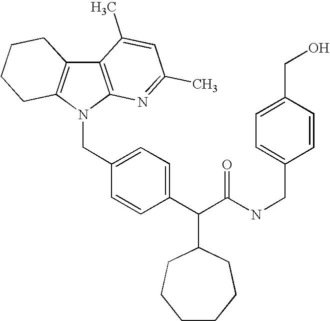 | 2-Cycloheptyl-2-[4-(2,4-dimethyl- 5,6,7,8-tetrahydro-pyrido[2,3-b]indol-9-ylmethyl)-phenyl]-N-(4- hydroxymethyl-benzyl)- acetamide |
| 39 | 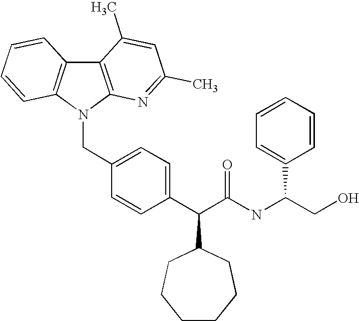 | 2-Cycloheptyl-2-[4-(2,4-dimethyl- pyrido[2,3-b]indol-9-ylmethyl)- phenyl]-N-(2-hydroxy-1-phenyl- ethyl)-acetamide |
| 40 | 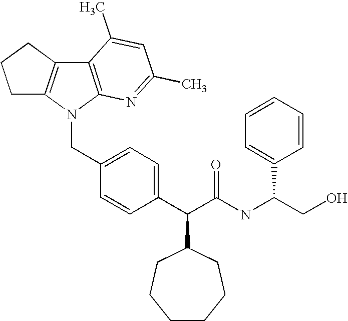 | 2-Cycloheptyl-2-[4-(4,6-dimethyl- 2,3-dihydro-1H-7,8-diaza- cyclopenta[a]inden-8-ylmethyl)- phenyl]-N-(2-hydroxy-1-phenyl- ethyl)-acetamide |
| 41 | 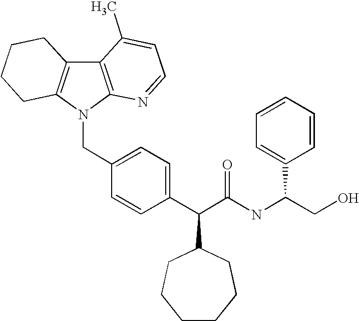 | 2-Cycloheptyl-N-(2-hydroxy-1- phenyl-ethyl)-2-[4-(4-methyl- 5,6,7,8-tetrahydro-pyrido[2,3- b]indol-9-ylmethyl)-phenyl]- acetamide |
| 42 | 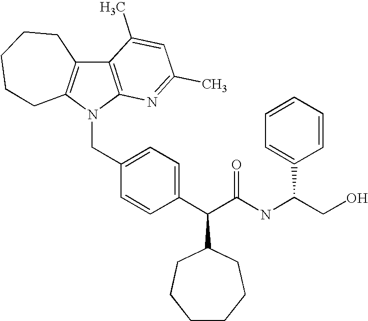 | 2-Cycloheptyl-2-[4-(2,4-dimethyl- 6,7,8,9-tetrahydro-5H-1,10-diaza- benzo[a]azulen-10-ylmethyl)- phenyl]-N-(2-hydroxy-1-phenyl- ethyl)-acetamide |
| 43 | 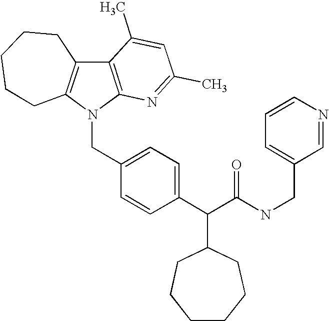 | 2-Cycloheptyl-2-[4-(2,4-dimethyl- 5,6,7,8-tetrahydro-pyrido[2,3- b]indol-9-ylmethyl)-phenyl]-N- pyridin-3-ylmethyl-acetamide |
| 44 | 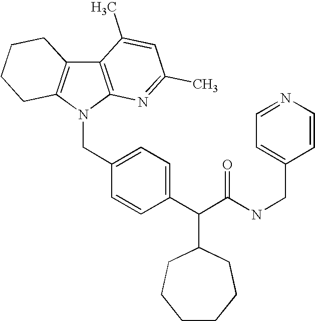 | 2-Cycloheptyl-2-[4-(2,4-dimethyl- 5,6,7,8-tetrahydro-pyrido[2,3- b]indol-9-ylmethyl)-phenyl]-N- pyridin-4-ylmethyl-acetamide |
| 45 | 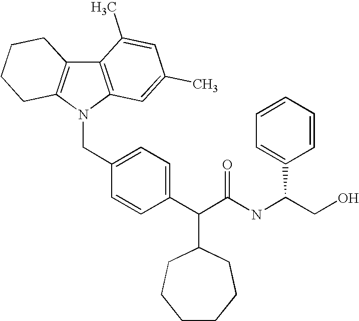 | 2-Cycloheptyl-2-[4-(5,7-dimethyl- 1,2,3,4-tetrahydro-carbazol-9- ylmethyl)-phenyl]-N-(2-hydroxy- 1-phenyl-ethyl)-acetamide |
| 46 | 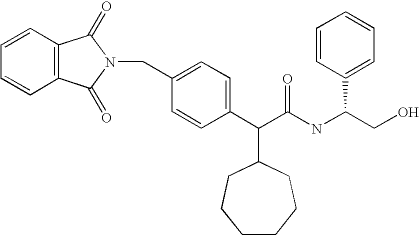 | 2-Cycloheptyl-2-[4-(5,7-dimethyl- 1,2,3,4-tetrahydro-carbazol-9- ylmethyl)-phenyl]-N-(2-hydroxy- 1-phenyl-ethyl)-acetamide |
| 47 | 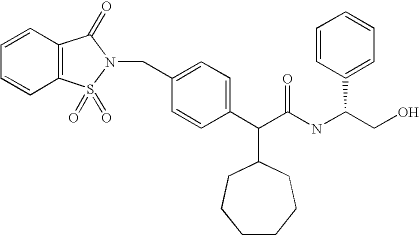 | 2-Cycloheptyl-N-(2-hydroxy-1- phenyl-ethyl)-2-[4-(1,1,3-trioxo- 1,3-dihydro-1,6- benzo[d]isothiazol-2-ylmethyl)- phenyl]-acetamide |
| 48 | 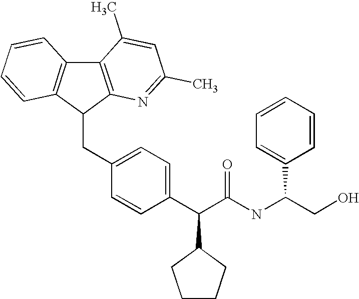 | (2S)-2-Cyclopentyl-2-[4-(2,4- dimethyl-pyrido[2,3-b]indol-9- ylmethyl)-phenyl]-N-(2-(1R)- hydroxy-1-phenyl-ethyl)- acetamide |
| 49 | 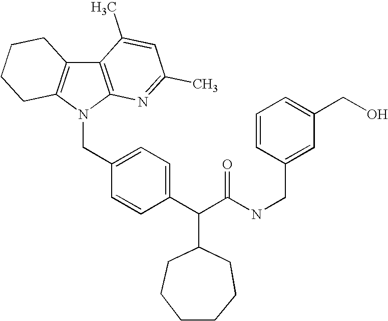 | 2-Cycloheptyl-2-[4-(2,4-dimethyl- 5,6,7,8-tetrahydro-pyrido[2,3- b]indol-9-ylmethyl)-phenyl]-N-(3- hydroxymethyl-benzyl)- acetamide |
| 50 | 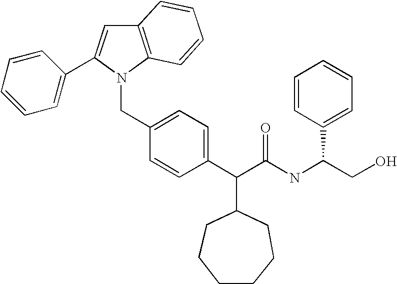 | 2-Cycloheptyl-N-(2-hydroxy-1- phenyl-ethyl)-2-[4-(2-phenyl- benzoimidazol-1-ylmethyl)- phenyl]-acetamide |
| 51 | 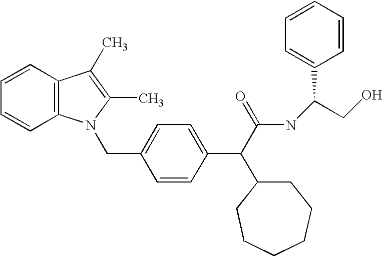 | 2-Cycloheptyl-2-[4-(2,3-dimethyl- indol-1-ylmethyl)-phenyl]-N-(2- hydroxy-1-phenyl-ethyl)- acetamide |
| 52 | 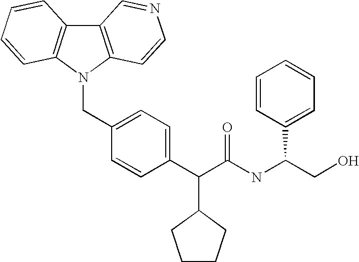 | 2-Cyclopentyl-N-(2-hydroxy-1- phenyl-ethyl)-2-(4-pyrido[4,3- b]indol-5-ylmethyl-phenyl)- acetamide |
| 53 | 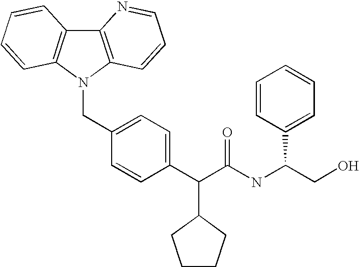 | 2-Cyclopentyl-N-(2-hydroxy-1-phenyl-ethyl)-2-(4-pyrido[3,2- b]indol-5-ylmethyl-phenyl)- acetamide |
| 54 | 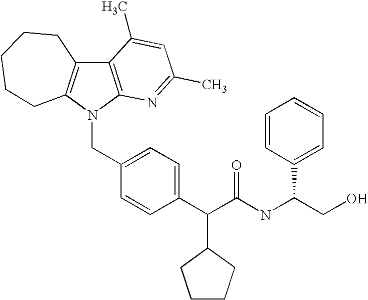 | 2-Cyclopentyl-2-[4-(2,4-dimethyl- 6,7,8,9-tetrahydro-5H-1,10-diaza- benzo[a]azulen-10-ylmethyl)- phenyl]-N-(2-hydroxy-1-phenyl- ethyl)-acetamide |
| 55 | 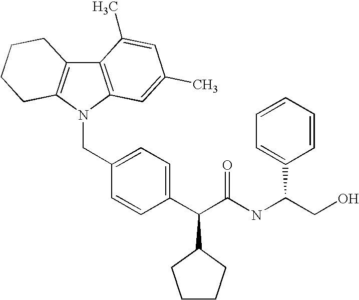 | 2-Cyclopentyl-2-[4-(5,7-dimethyl- 1,2,3,4-tetrahydro-carbazol-9- ylmethyl)-phenyl]-N-(2-hydroxy- 1-phenyl-ethyl)-acetamide |
| 56 | 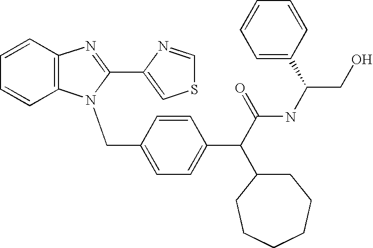 | 2-Cycloheptyl-N-(2-hydroxy-1- phenyl-ethyl)-2-[4-(2-thiazol-4-yl- benzoimidazol-1-ylmethyl)- phenyl]-acetamide |
| 57 | 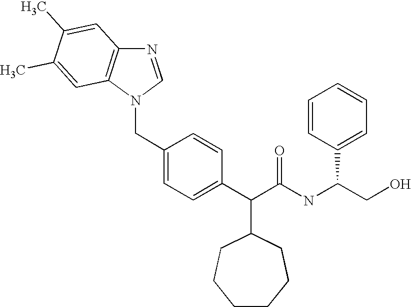 | 2-Cycloheptyl-2-[4-(5,6-dimethyl- benzoimidazol-1-ylmethyl)- phenyl]-N-(2-hydroxy-1-phenyl- ethyl)-acetamide |
| 58 | 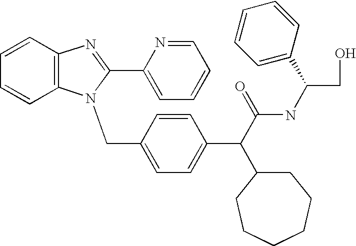 | 2-Cycloheptyl-N-(2-hydroxy-1- phenyl-ethyl)-2-[4-(2-pyridin-2-yl benzoimidazol-1-ylmethyl)- phenyl]-acetamide |
| 59 | 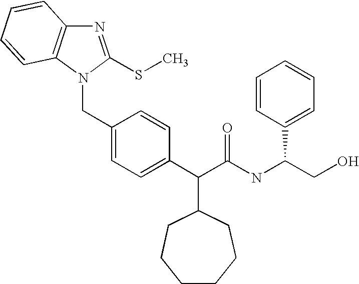 | 2-Cycloheptyl-N-(2-hydroxy-1- phenyl-ethyl)-2-[4-(2- methylsulfanyl-benzoimidazol-1- ylmethyl)-phenyl]-acetamide |
| 60 | 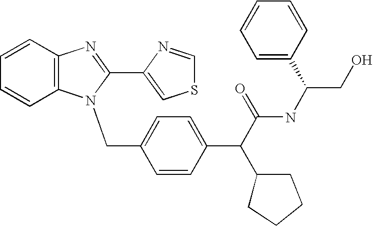 | 2-Cyclopentyl-N-(2-hydroxy-1- phenyl-ethyl)-2-[4-(2-thiazol-4-yl- benzoimidazol-1-ylmethyl)- phenyl]-acetamide |
| 61 | 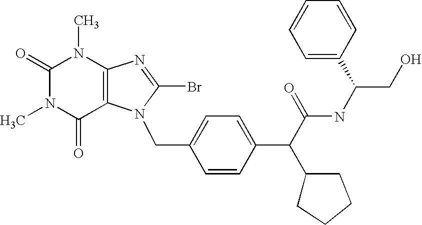 | 2-[4-(8-Bromo-1,3-dimethyl-2,6- dioxo-1,2,3,6-tetrahydro-purin-7- ylmethyl)-phenyl]-2-cyclopentyl- N-(2-hydroxy-1-phenyl-ethyl)- acetamide |
| 62 | 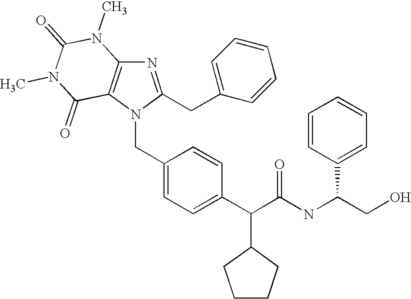 | 2-[4-(8-Benzyl-1,3-dimethyl-2,6- dioxo-1,2,3,6-tetrahydro-purin-7- ylmethyl)-phenyl]-2-cyclopentyl- N-(2-hydroxy-1-phenyl-ethyl)- acetamide |
| 63 | 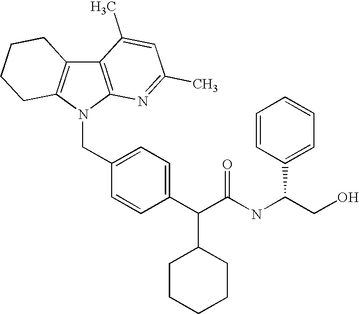 | 2-Cyclohexyl-2-[4-(2,4-dimethyl- 5,6,7,8-tetrahydro-pyrido[2,3- b]indol-9-ylmethyl)-phenyl]-N-(2- hydroxy-1-phenyl-ethyl)- acetamide |
| 64 | 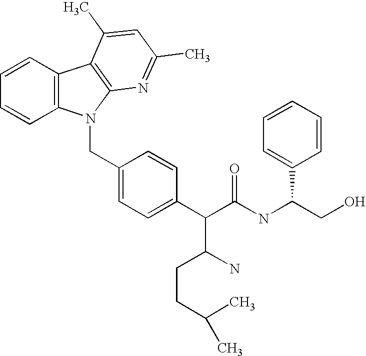 | 2-[4-(2,4-Dimethyl-pyrido[2,3- b]indol-9-ylmethyl)-phenyl]4- methyl-pentanoic acid (2-hydroxy 1-phenyl-ethyl)-amide |
| 65 | 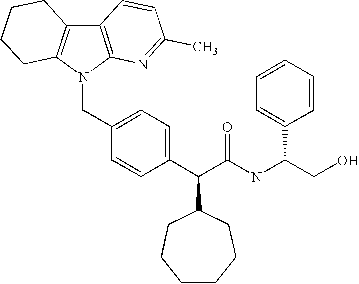 | 2-Cycloheptyl-N-(2-hydroxy-1- phenyl-ethyl)-2-[4-(2-methyl- 5,6,7,8-tetrahydro-pyrido[2,3- b]indol-9-ylmethyl)-phenyl]- acetamide |
| 66 | 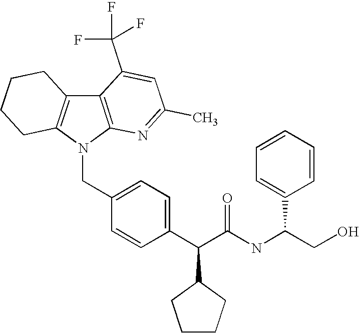 | 2-Cyclopentyl-N-(2-hydroxy-1- phenyl-ethyl)-2-[4-(2-methyl-4- trifluoromethyl-5,6,7,8-tetrahydro pyrido[2,3-b]indol-9-ylmethyl)- phenyl]-acetamide |
| 67 | 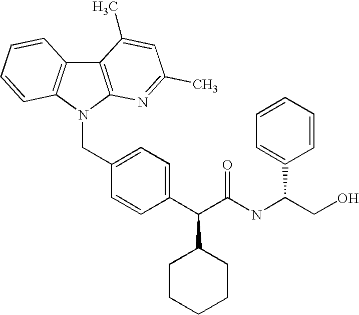 | 2-Cyclohexyl-2-[4-(2,4-dimethyl- pyrido[2,3-b]indol-9-ylmethyl)- phenyl]-N-(2-hydroxy-1-phenyl- ethyl)-acetamide |
| 68 | 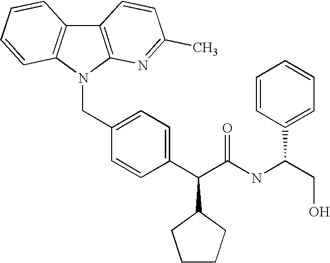 | 2-Cyclopentyl-N-(2-hydroxy-1- phenyl-ethyl)-2-[4-(2-methyl- pyrido[2,3-b]indol-9-ylmethyl)- phenyl]-acetamide |
| 69 | 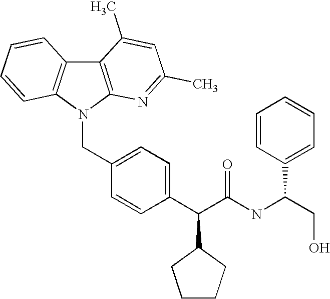 | 2-Cyclopentyl-N-(2-hydroxy-1- phenyl-ethyl)-2-[4-(4-methyl- pyrido[2,3-b]indol-9-ylmethyl)- phenyl]-acetamide |
| 70 | 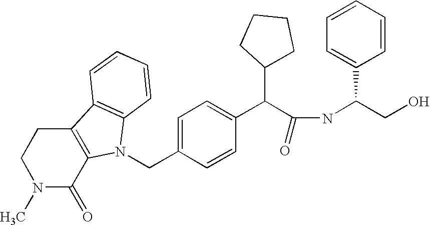 | 2-Cyclopentyl-N-(2-hydroxy-1- phenyl-ethyl)-2-[4-(2-methyl-1- oxo-1,2,3,4-tetrahydro-β-carbolin- 9-ylmethyl)-phenyl]-acetamide |
| 71 | 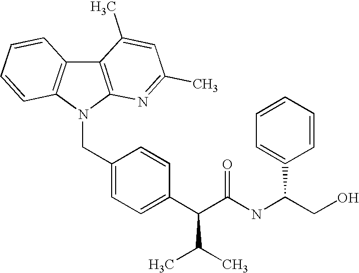 | 2-[4-(2,4-Dimethyl-pyrido[2,3- b]indol-9-ylmethyl)-phenyl]-N-(2- hydroxy-1-phenyl-ethyl)-3- methyl-butyramide |
| 72 | 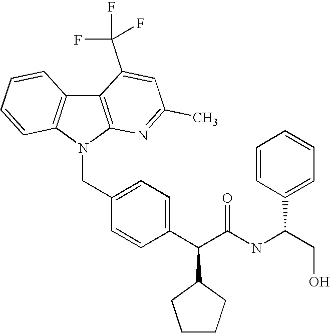 | 2-Cyclopentyl-N-(2-hydroxy-1- phenyl-ethyl)-2-[4-(2-methyl-4- trifluoromethyl-pyrido[2,3- b]indol-9-ylmethyl)-phenyl]- acetamide |
| 73 | 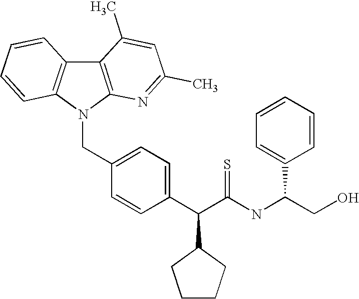 | 2-Cyclopentyl-2-[4-(2,4-dimethyl- pyrido[2,3-b]indol-9-ylmethyl)- phenyl]-N-(2-hydroxy-1-phenyl- ethyl)-thioacetamide |
| 74 | 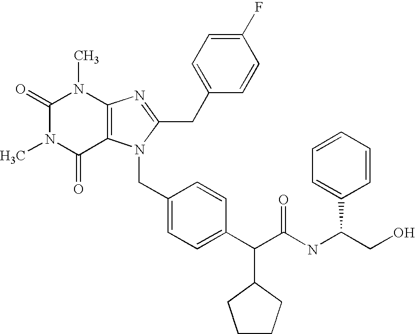 | 2-Cyclopentyl-2-{4-[8-(4-fluoro- benzyl)-1,3-dimethyl-2,6-dioxo- 1,2,3,6-tetrahydro-purin-7- ylmethyl]-phenyl}-N-(2-hydroxy- 1-phenyl-ethyl)-acetamide |
| 75 | 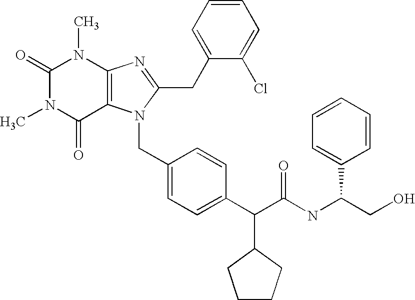 | 2-{4-[8-(2-Chloro-benzyl)-1,3- dimethyl-2,6-dioxo-1,2,3,6- tetrahydro-purin-7-ylmethyl]- phenyl}-2-cyclopentyl-N-(2- hydroxy-1-phenyl-ethyl)- acetamide |
| 76 | 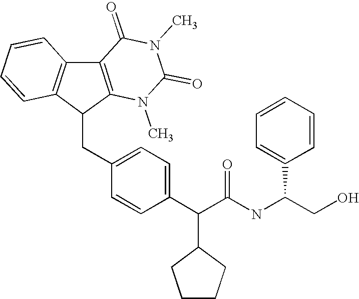 | 2-Cyclopentyl-2-[4-(1,3-dimethyl- 2,4-dioxo-1,2,3,4-tetrahydro- 1,3,9-triaza-fluoren-9-ylmethyl)- phenyl]-N-(2-hydroxy-1-phenyl- ethyl)-acetamide |
| 77 | 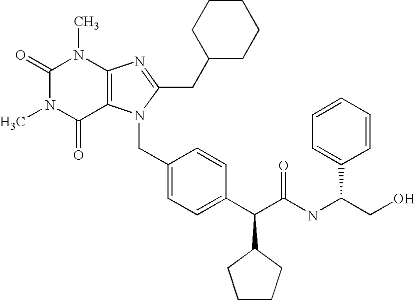 | 2-[4-(8-Cyclohexylmethyl-1,3- dimethyl-2,6-dioxo-1,2,3,6- tetrahydro-purin-7-ylmethyl)- phenyl]-2-cyclopentyl-N-(2- hydroxy-1-phenyl-ethyl)- acetamide |
| 78 | 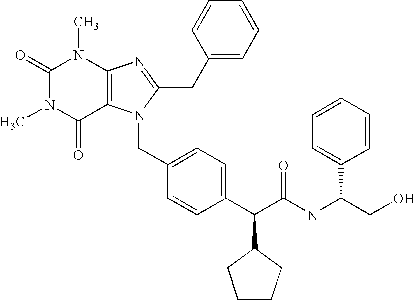 | 2-Cyclopentyl-2-[4-(1,3-dimethyl- 2,6-dioxo-8-m-tolyl-1,2,3,6- tetrahydro-purin-7-ylmethyl)- phenyl]-N-(2-hydroxy-1-phenyl- ethyl)-acetamide |
| 79 | 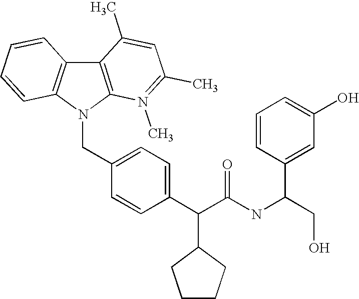 | 2-Cyclopentyl-2-[4-(2,4-dimethyl- pyrido[2,3-b]indol-9-ylmethyl)- phenyl]-N-[2-hydroxy-1-(3- hydroxy-phenyl)-ethyl]- acetamide |
| 80 | 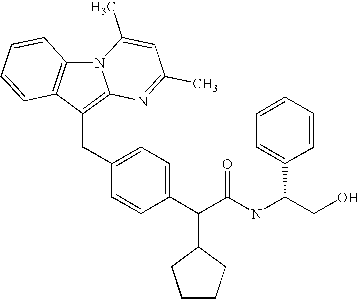 | (2S)-2-Cyclopentyl-2-[4-(2,4- dimethyl-pyrimido[1,2-a]indol-10 ylmethyl)-phenyl]-N-(2-(1R)- hydroxy-1-phenyl-ethyl)- acetamide |
| 81 | 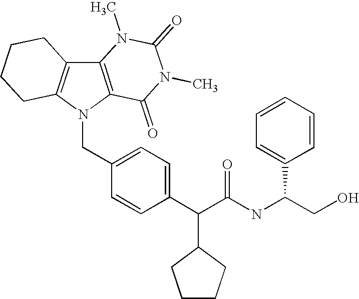 | 2-Cyclopentyl-2-[4-(2,4-dimethyl- 1,3-dioxo-1,2,3,4,5,6,7,8- octahydro-2,4,9-triaza-fluoren-9- ylmethyl)-phenyl]-N-(2-hydroxy- 1-phenyl-ethyl)-acetamide |
| 82 | 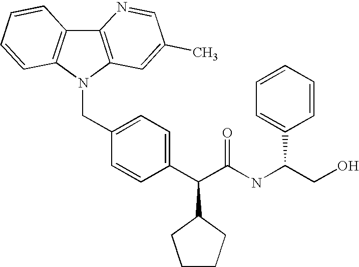 | 2-Cyclopentyl-N-(2-hydroxy-1- phenyl-ethyl)-2-[4-(3-methyl- pyrido[3,2-b]indol-5-ylmethyl)- phenyl]-acetamide |
| 83 | 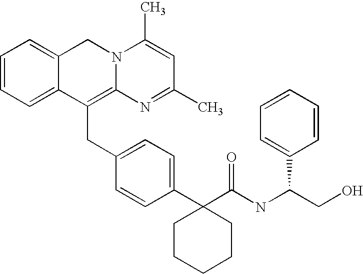 | 1-[4-(2,4-Dimethyl-pyrimido[1,2- a]indol-10-ylmethyl)-phenyl]- cyclohexanecarboxylic acid (2- hydroxy-1-phenyl-ethyl)-amide |
| 84 | 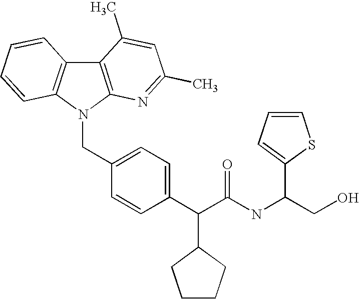 | 2-Cyclopentyl-2-[4-(2,4-dimethyl- pyrido[2,3-b]indol-9-ylmethyl)- phenyl]-N-(2-hydroxy-1-thiophen 2-yl-ethyl)-acetamide |
| 85 | 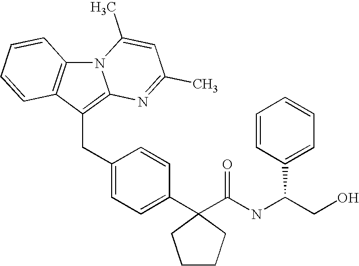 | 1-[4-(2,4-Dimethyl-pyrimido[1,2- a]indol-10-ylmethyl)-phenyl]- cyclopentanecarboxylic acid (2- hydroxy-1-phenyl-ethyl)-amide |
| 86 | 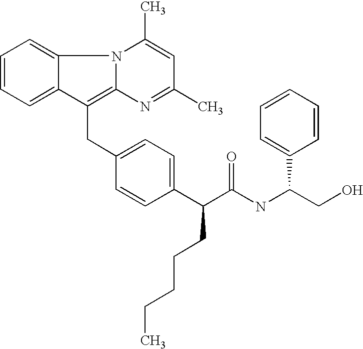 | 2-[4-(2,4-Dimethyl-pyrimido[1,2- a]indol-10-ylmethyl)-phenyl]- heptanoic acid (2-hydroxy-1- phenyl-ethyl)-amide |
| 87 | 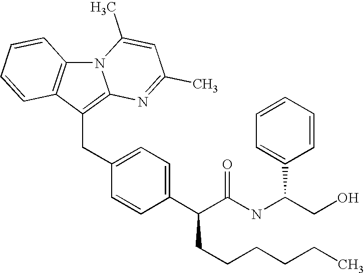 | 2-[4-(2,4-Dimethyl-pyrimido[1,2- a]indol-10-ylmethyl)-phenyl]- octanoic acid (2-hydroxy-1- phenyl-ethyl)-amide |
| 88 | 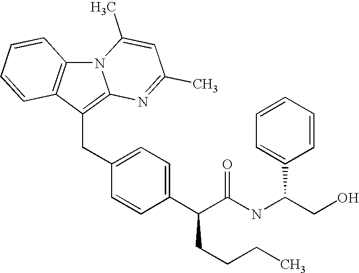 | 2-[4-(2,4-Dimethyl-pyrimido[1,2- a]indol-10-ylmethyl)-phenyl]- hexanoic acid (2-hydroxy-1- phenyl-ethyl)-amide |
| 89 | 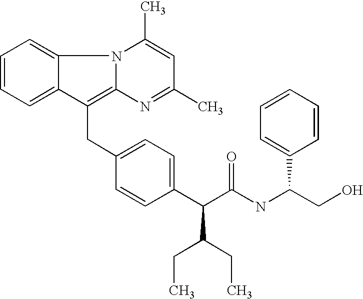 | 2-[4-(2,4-Dimethyl-pyrimido]1,2- a]indol-10-ylmethyl)-phenyl]-3- ethyl-pentanoic acid (2-hydroxy- 1-phenyl-ethyl)-amide |
| 90 | 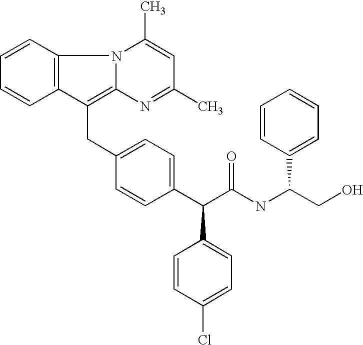 | 2-(4-Chloro-phenyl)-2-[4-(2,4- dimethyl-pyrimido[1,2-a]indol-10 ylmethyl)-phenyl]-N-(2-hydroxy- 1-phenyl-ethyl)-acetamide |
| 91 | 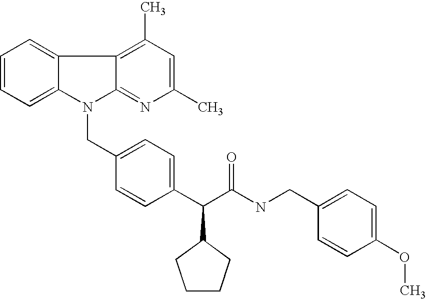 | 2-Cyclopentyl-2-[4-(2,4-dimethyl- pyrido[2,3-b]indol-9-ylmethyl)- phenyl]-N-(4-methoxy-benzyl)- acetamide |

Particularly preferred MTP inhibitors are the compounds listed in the following table:

| Ex. | | |
|---|---|---|
| No. | Structure | Name |
| 92 | 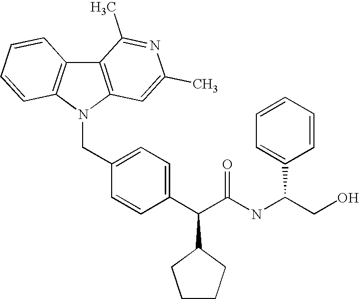 | 2-Cyclopentyl-2-[4-(1,3-dimethyl- pyrido[4,3-b]indol-5-ylmethyl)-phenyl]- N-(2-hydroxy-1-phenyl-ethyl)- acetamide |
| 93 | 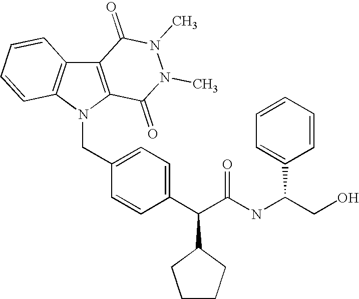 | 2-Cyclopentyl-2-[4-(2,3-dimethyl-1,4- dioxo-1,2,3,4-tetrahydro-2,3,9-triaza- fluoren-9-ylmethyl)-phenyl]-N-(2- hydroxy-1-phenyl-ethyl)-acetamide |
| 94 | 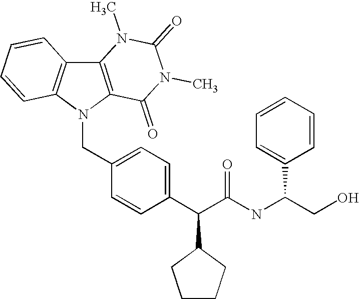 | 2-Cyclopentyl-2-[4-(2,4-dimethyl-1,3- dioxo-1,2,3,4-tetrahydro-2,4,9-triaza- fluoren-9-ylmethyl)-phenyl]-N-(2- hydroxy-1-phenyl-ethyl)-acetamide |
| 95 | 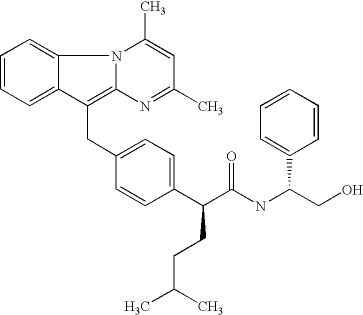 | 2-[4-(2,4-Dimethyl-pyrimido[1,2- a]indol-10-ylmethyl)-phenyl]-5-methyl- hexanoic acid (2-hydroxy-1-phenyl- ethyl)-amide |
| 96 | 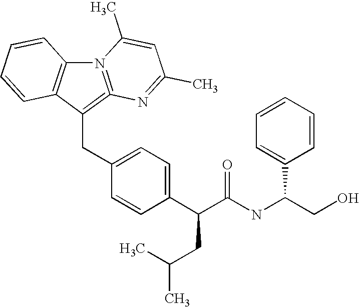 | 2-[4-(2,4-Dimethyl-pyrimido[1,2- a]indol-10-ylmethyl)-phenyl]-4-methyl- pentanoic acid (2-hydroxy-1-phenyl- ethyl)-amide |
| 97 | 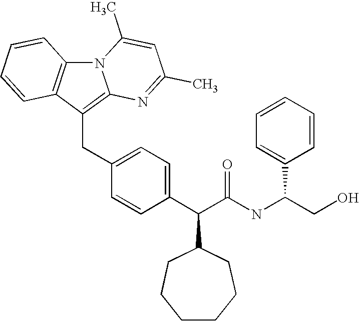 | 2-Cycloheptyl-2-[4-(2,4-dimethyl- pyrimido[1,2-a]indol-10-ylmethyl)- phenyl]-N-(2-hydroxy-1-phenyl-ethyl)- acetamide |
| 98 | 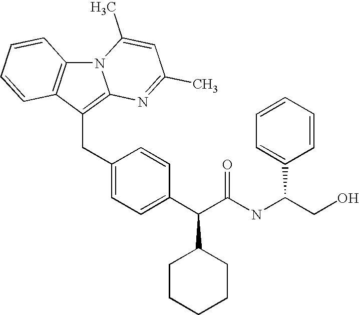 | 2-Cyclohexyl-2-[4-(2,4-dimethyl- pyrimido[1,2-a]indol-10-ylmethyl)- phenyl]-N-(2-hydroxy-1-phenyl-ethyl)- acetamide |
| 99 | 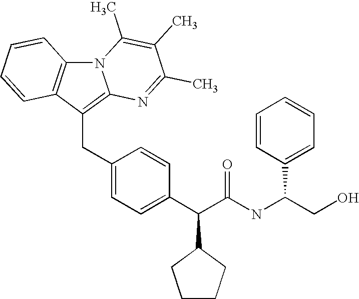 | 2-Cyclopentyl-N-(2-hydroxy-1-phenyl- ethyl)-2-[4-(2,3,4-trimethyl- pyrimido[1,2-a]indol-10-ylmethyl)- phenyl]-acetamide |
| 100 | 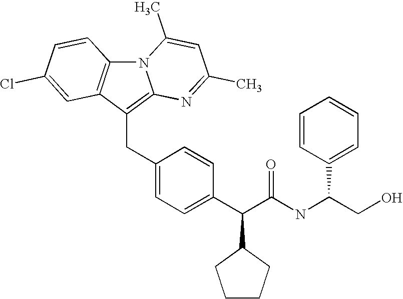 | 2-[4-(8-Chloro-2,4-dimethyl- pyrimido[1,2-a]indol-10-ylmethyl)- phenyl]-2-cyclopentyl-N-(2-hydroxy-1- phenyl-ethyl)-acetamide |
| 101 | 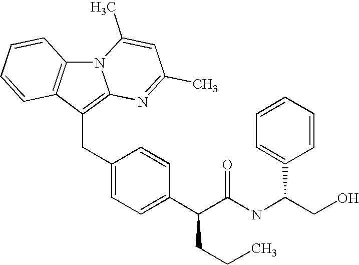 | 2-[4-(2,4-Dimethyl-pyrimido[1,2- a]indol-10-ylmethyl)-phenyl]-pentanoic acid (2-hydroxy-1-phenyl-ethyl)-amide |
| 102 | 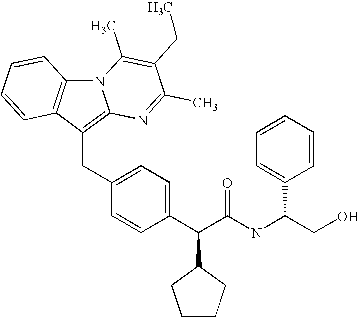 | 2-Cyclopentyl-2-[4-(3-ethyl-2,4- dimethyl-pyrimido[1,2-a]indol-10- ylmethyl)-phenyl]-N-(2-hydroxy-1- phenyl-ethyl)-acetamide |
| 103 | 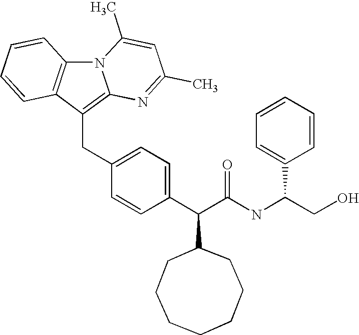 | 2-Cyclooctyl-2-[4-(2,4-dimethyl- pyrimido[1,2-a]indol-10-ylmethyl)- phenyl]-N-(2-hydroxy-1-phenyl-ethyl)- acetamide |
| 104 | 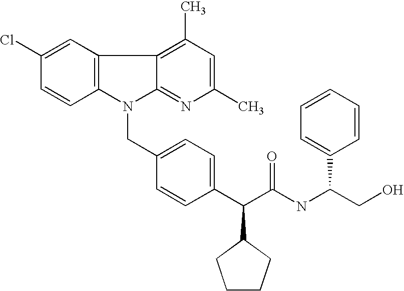 | 2-[4-(7-Chloro-2,4-dimethyl- pyrimido[1,2-a]indol-10-ylmethyl)- phenyl]-2-cyclopentyl-N-(2-hydroxy-1- phenyl-ethyl)-acetamide |
| 105 | 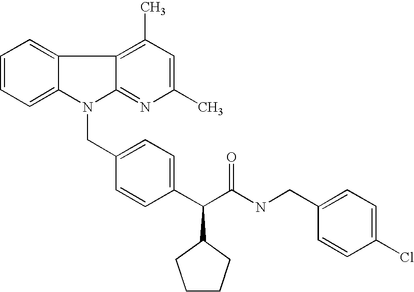 | N-(4-Chloro-benzyl)-2-cyclopentyl-2-[4 (2,4-dimethyl-pyrido[2,3-b]indol-9- ylmethyl)-phenyl]-acetamide |
| 106 | 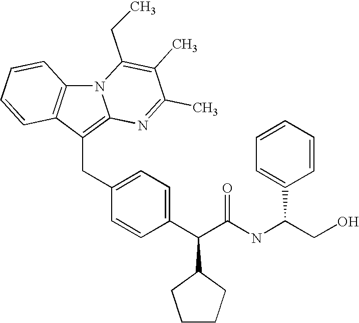 | 2-Cyclopentyl-2-[4-(4-ethyl-2,3- dimethyl-pyrimido[1,2-a]indol-10- ylmethyl)-phenyl]-N-(2-hydroxy-1- phenyl-ethyl)-acetamide |
| 107 | 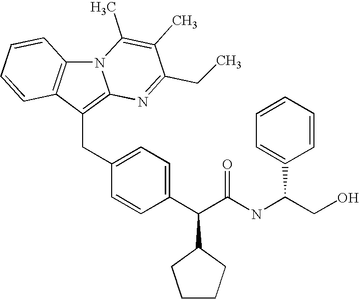 | 2-Cyclopentyl-2-[4-(2-ethyl-3,4- dimethyl-pyrimido[1,2-a]indol-10- ylmethyl)-phenyl]-N-(2-hydroxy-1- phenyl-ethyl)-acetamide |
| 108 | 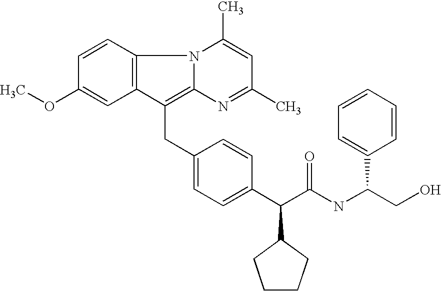 | 2-Cyclopentyl-N-(2-hydroxy-1-phenyl- ethyl)-2-[4(8-methoxy-2,4-dimethyl- pyrimido[1,2-a]indol-10-ylmethyl)-phenyl]-acetamide |
| 109 | 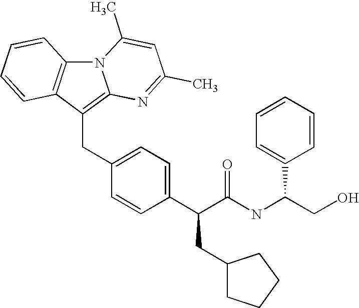 | 3-Cyclopentyl-2-[4-(2,4-dimethyl- pyrimido[1,2-a]indol-10-ylmethyl)- phenyl]-N-(2-hydroxy-1-phenyl-ethyl)-propionamide |
| 110 | 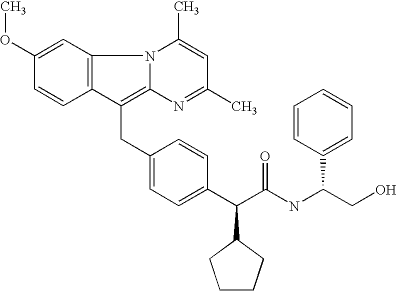 | 2-Cyclopentyl-N-(2-hydroxy-1-phenyl- ethyl)-2-[4-(7-methoxy-2,4-dimethyl- pyrimido[1,2-a]indol-10-ylmethyl)- phenyl]-acetamide |
| 111 | 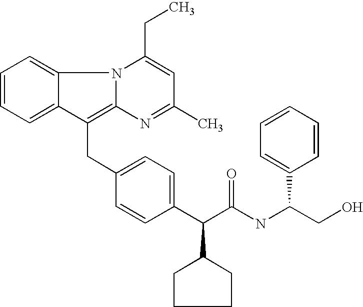 | 2-Cyclopentyl-2-[4-(4-ethyl-2-methyl- pyrimido[1,2-a]indol-10-ylmethyl)- phenyl]-N-(2-hydroxy-1-phenyl-ethyl)- acetamide |
| 112 | 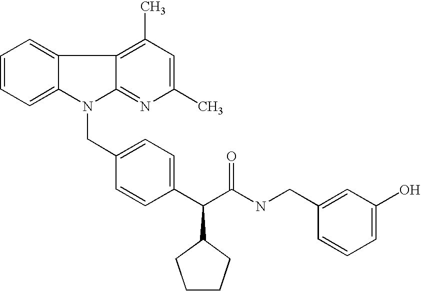 | 2-Cyclopentyl-2-[4-(2,4-dimethyl- pyrido[2,3-b]indol-9-ylmethyl)-phenyl]- N-(3-hydroxy-benzyl)-acetamide |
| 113 | 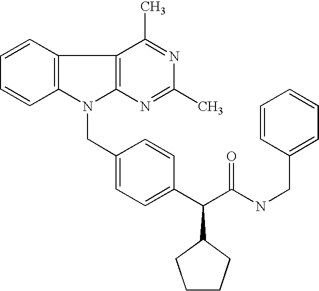 | N-Benzyl-2-cyclopentyl-2-[4-(2,4- dimethyl-1,3,9-triaza-fluoren-9- ylmethyl)-phenyl]-acetamide |
| 114 | 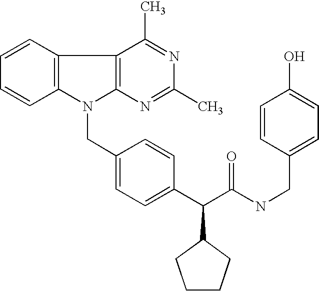 | 2-Cyclopentyl-2-[4-(2,4-dimethyl-1,3,9- triaza-fluoren-9-ylmethyl)-phenyl]-N-(4- hydroxy-benzyl)-acetamide |
| 115 | 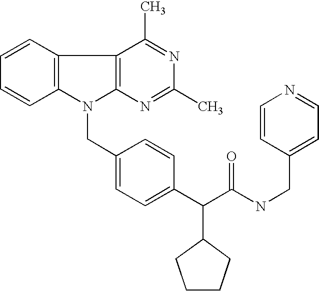 | 2-Cyclopentyl-2-[4-(2,4-dimethyl-1,3,9- triaza-fluoren-9-ylmethyl)-phenyl]-N- pyridin-4-ylmethyl-acetamide |
| 116 | 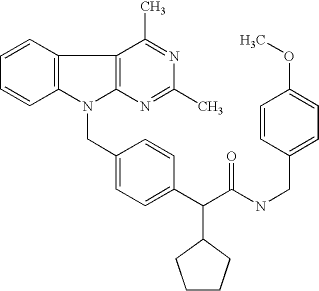 | 2-Cyclopentyl-2-[4-(2,4-dimethyl-1,3,9- triaza-fluoren-9-ylmethyl)-phenyl]-N-(4--nl methoxy-benzyl)-acetamide |
| 117 | 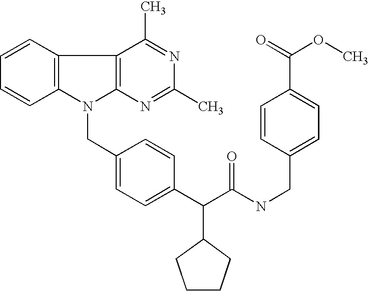 | 4-({2-Cyclopentyl-2-[4-(2,4-dimethyl- 1,3,9-triaza-fluoren-9-ylmethyl)- phenyl]-acetylamino}-methyl)-benzoic acid methyl ester |
| 118 | 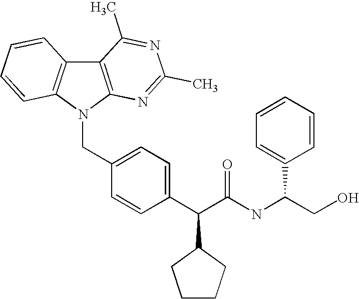 | 2-Cyclopentyl-2-[4-(2,4-dimethyl-1,3,9- triaza-fluoren-9-ylmethyl)-phenyl]-N-(2- hydroxy-1-phenyl-ethyl)-acetamide |
| 119 | 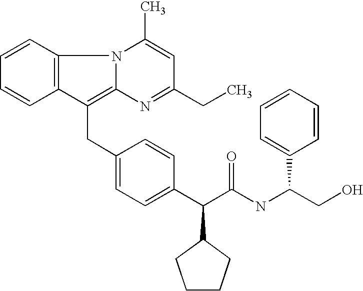 | 2-Cyclopentyl-2-[4-(2-ethyl-4-methyl- pyrimido[1,2-a]indol-10-ylmethyl)- phenyl]-N-(2-hydroxy-1-phenyl-ethyl)- acetamide |

The MTP inhibitors can be employed according to the invention for example for the treatment and/or prophylaxis of disorders associated in particular with impairments of postprandial lipoprotein or lipid metabolism. Such impairments mean herein: accumulation and/or prolonged persistence of ppTRL, chylomicrons and cholesterol-rich remnants in the plasma, and elevated or more persistent postprandial plasma lipid levels.

Disorders associated therewith are, for example, besides cardiovascular diseases, primarily manifestations of neurodegenerative deficits. Those which should be particularly mentioned in this connection are neuropathological changes in the brain and their sequelae: neurodegeneration such as, for example, associated with Alzheimer's disease, progressive atrophy of the brain, morphological changes in the brain during the normal aging process (presenile dementia), impairment of the cortical cholinergic system, memory impairments, orientation impairments, aphasia, wordfinding impairments, agnosia, apraxia, euphoria, depression, Binswanger's disease, Pick's disease, Niemann-Pick disease, cerebrovascular insufficiency.

Examples of cardiovascular diseases which are associated with impairments of postprandial lipoprotein or lipid metabolism and which may be mentioned here are: arteriosclerosis, stroke, angina, disorders of the coronary vessels of the heart, especially of the arterial coronary vessels, heart failure, primary and secondary myocardial infarction, pathological changes in the vessel wall, impairments of blood flow and of the microcirculation.

Likewise associated with elevated and more persistent ppTRL levels are impairments of carbohydrate metabolism such as, for example, insulin resistance, IGT (impaired glucose tolerance), diabetes, especially type 2 diabetes, metabolic syndrome. These diseases can therefore also be treated with MTP inhibitors.

It may be advantageous to employ the MTP inhibitors in combination with other suitable active ingredients. Those which may be mentioned are: acetylcholinesterase inhibitors, e.g. metrifonate, tacrine and donepezil, substances which inhibit abnormal cleavage of amyloid precursor protein, estrogens such as, for example, estradiol, synthetic estrogen receptor agonists, vitamin E,

Deliberate diminution of plasma ppTRL levels also leads to an improved tolerability of the MTP inhibitors. Side effects which may occur at high dosages are avoided in particular through lower dosages. In addition, with low dosages only slight or no effects due to the mechanism are to be expected in the liver; consequently, no side effects due to the mechanism can be induced in the liver either.

The MTP inhibitors are preferably employed in human medicine, but are also suitable for veterinary medicine, in particular for the treatment of mammals.

The combinations of the invention can be administered parenterally or, preferably, orally.

The MTP inhibitors can be converted in a known manner into conventional formulations, which may be liquid or, preferably, solid formulations. Examples are tablets, coated tablets, pills, capsules, granules, aerosols, syrups, emulsions, suspensions, solutions.

The MTP inhibitors are [lacuna] on oral administration preferably in dosages of from 0.01 to 20 mg/kg, in particular 0.1 to 5 mg, of active ingredient per kg of the patient's weight.

It may, where appropriate, be necessary to deviate from the stated amounts, in particular as a function of the body weight and nature of the administration route, of the individual behavior toward the medicament, of the nature of its formulation and the time or interval over which administration takes place. Thus, in some cases, less than the aforementioned minimum amount may be sufficient, whereas in other cases the stated upper limit must be exceeded. In the event of administration of relatively large amounts, it may be advisable to divide these into a plurality of single doses during the day.

The solid oral dosage forms mentioned herein are produced by general standard processes. Ingredients are those which are pharmaceutically accepted and physiologically unobjectionable, for example: as fillers cellulose derivatives (e.g. microcrystalline cellulose), sugars (e.g. lactose), sugar alcohols (e.g. mannitol, sorbitol), inorganic fillers (e.g. calcium phosphates), binders (e.g. polyvinylpyrrolidone, gelatin, starch derivatives and cellulose derivatives), and all other excipients required to produce pharmaceutical formulations with the desired properties, e.g. lubricants (magnesium stearate), e.g. disintegrants (e.g. crosslinked polyvinylpyrrolidone, sodium carboxymethylcellulose), e.g. wetting agents (e.g. sodium lauryl sulfate), e.g. release-slowing agents (e.g. cellulose derivatives, polyacrylic acid derivatives), e.g. stabilizers, e.g. flavorings, e.g. colored pigments.

Liquid formulations are likewise produced by standard methods using pharmaceutically usable excipients and comprise the active ingredient or the two active ingredients either in solution or in suspension. Typical volumes of these pharmaceutical preparations administered are 1 to 10 ml. Examples of excipients in these liquid formulations are: solvents (e.g. water, alcohol, natural and synthetic oils, e.g. medium chain-length triglcerides), solubilizers (e.g. glycerol, glycol derivatives), wetting agents (e.g. polysorbate, sodium lauryl sulfate), and other excipients required to produce pharmaceutical formulations with the desired properties, e.g. viscosity-increasing agents, e.g. pH correctives, e.g. sweeteners and flavorings, e.g. antioxidants, e.g. stabilizers, e.g. preservatives.

The main constituents of the shells of capsule formulations are, for example, gelatin or hydroxypropylmethylcellulose.

Pharmaceutical excipients familiar to the skilled worker are, for example, also described in the following handbook: “Handbook of Pharmaceutical Excipients”, Wade, A. & Weller, P. J., American Pharmaceutical Association, Washington, 2nd edition 1994.

### EXAMPLE

Long-term Feeding Experiment with ApoE-Knockout Mice

ApoE-knockout mice received a fat-containing diet (0.15% cholesterol, 21.4% crude fat, 19% casein; “Western diet”). The compound of Example 48 was administered in a proportion of 5 ppm with the feed to the treated group, while the control group received the food without active ingredient. After 13 months, over half of the untreated mice were dead, whereas 23 of 25 animals in the treated group were still alive.

It is known from the literature that the ApoE-knockout mice used die from myocardial infarctions and manifestations of neuronal degeneration [Caligiuri, G., et al., Proc. Natl. Acad. Sci. USA, 96, 6920-6924 (1999); Walker, L. C. Am.J.Pathol., 151, (5), 1371-1377 81997)].

1. The use of MTP inhibitors for producing medicaments for reducing the ppTRL plasma level.

2. The use of MTP inhibitors for producing medicaments for diminishing intestinal cholesterol absorption.

3. The use as claimed in claim 1 or 2, characterized in that the MTP inhibitor is employed in a concentration with which no more than a slight reduction in the fasting levels of plasma triglycerides or of plasma cholesterol is induced.

4. The use as claimed in any of claims 1 to 3, characterized in that the MTP inhibitor employed is a compound of the general formula (A1) 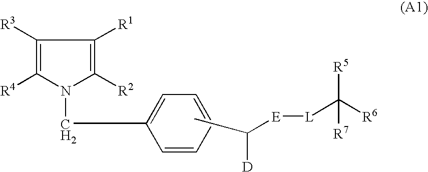
in which
R¹ and R² together form, with inclusion of the double bond connecting them, a phenyl or pyridyl ring or a ring of the formula 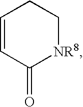
in which
R⁸ is hydrogen or straight-chain or branched alkyl having up to 4 carbon atoms,
R³ and R⁴ together form, with inclusion of the double bond connecting them, a phenyl ring or a 4- to 8-membered cycloalkene or oxocycloalkene residue, where all ring systems mentioned under R¹/R² and R³/R⁴ optionally have up to 3 identical or different halogen, trifluoromethyl, carboxyl, hydroxyl substituents, straight-chain or branched alkoxy or alkoxycarbonyl substituents each having up to 6 carbon atoms, or straight-chain or branched alkyl substituents which have up to 6 carbon atoms and which in turn may be substituted by hydroxyl or by straight-chain or branched alkoxy having up to 4 carbon atoms,
D is hydrogen, cycloalkyl having 4 to 12 carbon atoms or is straight-chain or branched alkyl having up to 12 carbon atoms,
E is the —CO— or —CS— group,
L is an oxygen or sulfur atom or is a group of the formula —NR⁹,
in which
R⁹ is hydrogen or straight-chain or branched alkyl which has up to 6 carbon atoms and which is optionally substituted by hydroxyl or phenyl,
R⁵ is phenyl or is a 5- to 7-membered saturated or unsaturated heterocycle having up to 3 heteroatoms from the series S, N and/or O, where the rings optionally have up to 3 identical or different nitro, carboxyl, halogen, cyano substituents or straight-chain or branched alkenyl or alkoxycarbonyl substituents each having up to 6 carbon atoms or straight-chain or branched alkyl substituents which have up to 6 carbon atoms and which are optionally substituted by hydroxyl, carboxyl or by straight-chain or branched alkoxy or alkoxycarbonyl each having up to 6 carbon atoms, and/or the rings are optionally substituted by a group of the formula —OR¹⁰ or —NR¹¹R¹²,
in which
R¹⁰ is hydrogen or straight-chain or branched alkyl or alkenyl each having up to 6 carbon atoms,
R¹¹ and R¹² are identical or different and are phenyl, hydrogen or straight-chain or branched alkyl having up to 6 carbon atoms, or straight-chain or branched acyl which has up to 8 carbon atoms and which is optionally substituted by a group of the formula —NR¹³R¹⁴,
in which
R¹³ and R¹⁴ are identical or different and are hydrogen or straight-chain or branched acyl having up to 8 carbon atoms,
R⁶ is hydrogen, carboxyl or is straight-chain or branched alkoxycarbonyl having up to 5 carbon atoms, or is straight-chain or branched alkyl which has up to 6 carbon atoms and which is optionally substituted by hydroxyl or by a group of the formula —O—CO—R¹⁵,
in which
R¹⁵ is phenyl which optionally has up to 3 identical or different halogen, hydroxyl substituents or straight-chain or branched alkyl substituents having up to 5 carbon atoms, or straight-chain or branched alkyl or alkenyl which each have up to 22 carbon atoms and which are optionally substituted by a group of the formula —OR¹⁶,
in which
R¹⁶ is hydrogen, benzyl, triphenylmethyl or straight-chain or branched acyl having up to 6 carbon atoms,
R⁷ is hydrogen or
R⁶ and R⁷ together are the group of the formula ═O,
or of the general formula (A2) 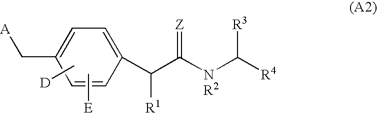
in which
A is a radical of the formula 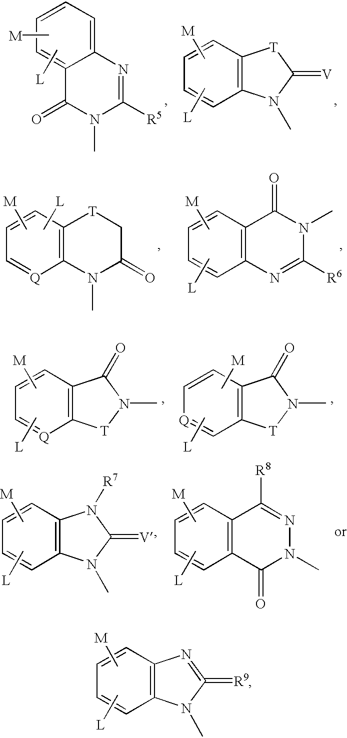
in which
L and M are identical or different and are hydrogen, halogen, trifluoromethyl, carboxyl, cycloalkyl having 3 to 6 carbon atoms, hydroxyl, phenyl or straight-chain or branched alkyl, alkoxycarbonyl or alkoxy each having up to 6 carbon atoms,
Q is a nitrogen atom or the —CH group,
T is a group of the formula —SO₂ or —CO or an oxygen or sulfur atom,
V is an oxygen or sulfur atom,
R⁵, R⁶, R⁷ and R⁸ are identical or different and are hydrogen, straight-chain or branched alkyl having up to 6 carbon atoms, benzyl or phenyl, which are optionally substituted by halogen or by straight-chain or branched alkyl having up to 6 carbon atoms,
R⁹ is trifluoromethyl, benzyl or a 5- to 7-membered, optionally benzo-fused heterocycle having up to 3 heteroatoms from the series S, N and/or O and optionally having up to 3 identical or different halogen, phenyl, hydroxyl substituents or straight-chain or branched alkyl or alkoxy substituents each having up to 4 carbon atoms, or is a group of the formula —S(O)ₐ—R¹⁰,
in which
a is a number 0, 1 or 2,
R¹⁰ is straight-chain or branched alkyl or alkenyl which each have up to 8 carbon atoms and which are optionally substituted by straight-chain or branched acyl having up to 6 carbon atoms or by aryl or aroyl each of which have up to 10 carbon atoms and which may in turn have up to 2 identical or different halogen, trifluoromethyl substituents or straight-chain or branched acyl substituents having up to 5 carbon atoms, or is aryl which has 6 to 10 carbon atoms and which is optionally substituted by halogen, hydroxyl, trifluoromethyl or straight-chain or branched alkyl or alkoxy each having up to 5 carbon atoms,
D and E are identical or different and are hydrogen, halogen, trifluoromethyl, hydroxyl, carboxyl or straight-chain or branched alkyl, alkoxy or alkoxycarbonyl each having up to 6 carbon atoms,
Z is an oxygen or sulfur atom,
R¹ is cycloalkyl having 3 to 10 carbon atoms or is straight-chain or branched alkyl having 1 to 10 carbon atoms, or is phenyl which optionally has up to 2 identical or different halogen, nitro, cyano, hydroxyl substituents or straight-chain or branched alkyl or alkoxy substituents each having up to 4 carbon atoms,
R² is hydrogen or straight-chain or branched alkyl having up to 3 carbon atoms,
R³ is hydrogen or straight-chain or branched alkyl having up to 5 carbon atoms, or is cycloalkyl having 3 to 7 carbon atoms, or is phenyl or a 5- to 7-membered aromatic heterocycle having up to 3 heteroatoms from the series S, N and/or O, which optionally have up to 3 identical or different halogen, nitro, phenyl, hydroxyl substituents or straight-chain or branched alkyl or alkoxy substituents having up to 6 carbon atoms,
R⁴ is hydrogen or is a group of the formula —CH₂—OH or CH₂O—CO—R¹¹,
in which
R¹¹ is hydrogen, straight-chain or branched alkyl having up to 8 carbon atoms or phenyl which optionally has up to 3 identical or different halogen, hydroxyl, cyano substituents or straight-chain or branched alkyl or alkoxy substituents each having up to 4 carbon atoms,
or of the general formula (A3) 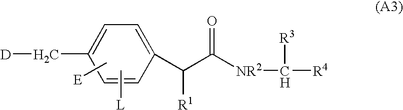
in which
D is a radical of the formula 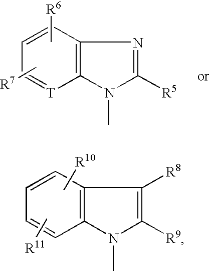
in which
T is a nitrogen atom or the —CH group,
R⁶, R⁷, R¹⁰ and R¹¹ are identical or different and are hydrogen, trifluoromethyl, halogen or straight-chain or branched alkyl or alkoxy each having up to 6 carbon atoms,
R⁵, R⁸ and R⁹ are identical or different and are hydrogen, cycloalkyl having 3 to 6 carbon atoms, phenyl, straight-chain or branched alkoxycarbonyl having up to 6 carbon atoms or straight-chain or branched alkyl which has up to 6 carbon atoms and which is optionally substituted by halogen, or, in the case where T is a nitrogen atom, R⁵ can also be a benzyl,
E and L are identical or different and are hydrogen, halogen, trifluoromethyl, hydroxyl, carboxyl or straight-chain or branched alkyl, alkoxy or alkoxycarbonyl each having up to 6 carbon atoms,
R¹ is cycloalkyl having 3 to 10 carbon atoms or is straight-chain or branched alkyl having 1 to 10 carbon atoms, or is phenyl which optionally has up to 2 identical or different halogen, cyano, hydroxyl substituents or straight-chain or branched alkyl or alkoxy substituents each having up to 4 carbon atoms,
R² is hydrogen or straight-chain or branched alkyl having up to 3 carbon atoms,
R³ is hydrogen or straight-chain or branched alkyl having up to 5 carbon atoms, or is cycloalkyl having 3 to 7 carbon atoms, or is phenyl or a 5- to 7-membered aromatic heterocycle having up to 3 heteroatoms from the series S, N and/or O, which optionally have up to 3 identical or different halogen, nitro, phenyl, hydroxyl substituents or straight-chain or branched alkyl or alkoxy substituents having up to 6 carbon atoms,
R⁴ is hydrogen or is a group of the formula —CH₂—OH or CH₂O—CO—R¹²,
in which
R¹² is hydrogen, straight-chain or branched alkyl having up to 8 carbon atoms or phenyl which optionally has up to 3 identical or different halogen, hydroxyl, cyano substituents or straight-chain or branched alkyl or alkoxy substituents each having up to 4 carbon atoms,
or of the general formula (A4) 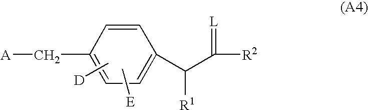
in which
A is a radical of the formula 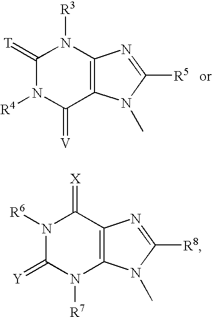
in which
R³, R⁴, R⁶ and R⁷ are identical or different and are hydrogen, cycloalkyl having 3 to 7 carbon atoms or aryl having 6 to 10 carbon atoms, or straight-chain or branched alkyl or alkenyl which each have up to 8 carbon atoms and which are optionally substituted by halogen, hydroxyl or aryl having 6 to 10 carbon atoms,
T, V, X and Y are identical or different and are an oxygen or sulfur atom,
R⁵ and R⁸ are identical or different and are hydrogen, halogen, cycloalkyl having 3 to 8 carbon atoms or straight-chain or branched alkyl or alkenyl which each have up to 8 carbon atoms and which are optionally substituted by cycloalkyl having 3 to 8 carbon atoms or by a 5- to 6-membered aromatic, optionally benzo-fused heterocycle having up to 3 heteroatoms from the series S, N and/or O, or by aryl having 6 to 10 carbon atoms, where the rings in turn may have up to 3 identical or different 5- to 6-membered aromatic heterocyclic substituents having up to 3 heteroatoms from the series S, N and/or O, or phenyl, benzyl, halogen, hydroxyl, carboxyl substituents or straight-chain or branched alkyl, alkoxy or alkoxycarbonyl substituents each having up to 6 carbon atoms, or aryl having 6 to 10 carbon atoms or a 5- to 7-membered aromatic, optionally benzo-fused heterocycle having up to 3 heteroatoms from the series S, N and/or O, which optionally have up to 3 identical or different halogen, phenyl, trifluoromethyl, hydroxyl, carboxyl substituents or straight-chain or branched alkyl, alkoxy or alkoxycarbonyl substituents each having up to 6 carbon atoms, or a substituent group of the formula —(CO)ₐ—NR⁹R¹⁰,
in which
a is a number 0 or 1,
R⁹ and R¹⁰ are identical or different and are hydrogen, phenyl or straight-chain or branched alkyl or acyl each having up to 5 carbon atoms,
D and E are identical or different and are hydrogen, halogen, trifluoromethyl, hydroxyl, carboxyl or are straight-chain or branched alkyl, alkoxy or alkoxycarbonyl each having up to 6 carbon atoms,
R¹ is hydrogen or cycloalkyl having 3 to 8 carbon atoms, or is straight-chain or branched alkyl or alkenyl which each have up to 8 carbon atoms and which are optionally substituted by cycloalkyl having 3 to 6 carbon atoms, phenyl or by a 5- to 6-membered aromatic heterocycle having up to 3 heteroatoms from the series S, N and/or O, or is phenyl or a 5- to 6-membered aromatic heterocycle having up to 3 heteroatoms from the series S, N and/or O, where the ring systems optionally have up to 3 identical or different halogen, phenyl, trifluoromethyl substituents or straight-chain or branched alkyl or alkoxy substituents each having up to 5 carbon atoms, hydroxyl substituents or a substituent group of the formula —NR¹¹R¹²,
in which
R¹¹ and R¹² have the abovementioned meaning of R⁹ and R¹⁰ and are identical to or different from the latter,
L is an oxygen or sulfur atom,
R² is mercapto, hydroxyl, straight-chain or branched alkoxy having up to 8 carbon atoms or the group of the formula 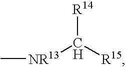
in which
R¹³ is hydrogen or straight-chain or branched alkyl having up to 4 carbon atoms,
R¹⁴ is hydrogen, phenyl or a 5- to 6-membered aromatic heterocycle having up to 3 heteroatoms from the series S, N and/or O,
R¹⁵ is hydrogen or straight-chain or branched alkyl which has up to 8 carbon atoms and is optionally substituted by hydroxyl,
or of the general formula (A5) 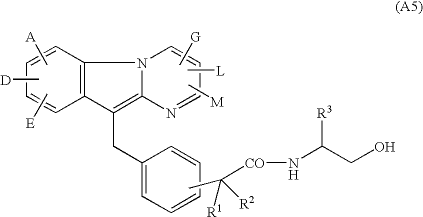
in which
A, D, E, G, L and M are identical or different and are hydrogen, halogen, trifluoromethyl, carboxyl, hydroxyl, straight-chain or branched alkoxy or alkoxycarbonyl each having up to 6 carbon atoms or straight-chain or branched alkyl which has up to 6 carbon atoms and which in turn may be substituted by hydroxyl or by straight-chain or branched alkoxy having up to 4 carbon atoms,
R¹ and R² are identical or different and are hydrogen, cycloalkyl having 3 to 8 carbon atoms or straight-chain or branched alkyl which has up to 10 carbon atoms and which is optionally substituted by cycloalkyl having 3 to 6 carbon atoms, or are phenyl which is optionally substituted by halogen or trifluoromethyl, or
R¹ and R² form, together with the carbon atom, a 4-8-membered cycloalkyl ring,
and
R³ is phenyl which optionally has up to 3 identical or different nitro, carboxyl, halogen, cyano substituents or straight-chain or branched alkenyl or alkoxycarbonyl substituents each having up to 6 carbon atoms, or straight-chain or branched alkyl substituents which have up to 6 carbon atoms and which are optionally substituted by hydroxyl, carboxyl or by straight-chain or branched alkoxy or alkoxycarbonyl each having up to 6 carbon atoms, and/or is optionally substituted by a group of the formula —OR⁴ or —NR⁵R⁶,
in which
R⁴ is hydrogen or straight-chain or branched alkyl or alkenyl each having up to 6 carbon atoms,
R⁵ and R⁶ are identical or different and are phenyl, hydrogen or straight-chain or branched alkyl having up to 6 carbon atoms, or straight-chain or branched acyl which has up to 8 carbon atoms and which is optionally substituted by a group of the formula —NR⁷R⁸,
in which
R⁷ and R⁸ are identical or different and are hydrogen or straight-chain or branched acyl having up to 8 carbon atoms,
or of the general formula (A6) 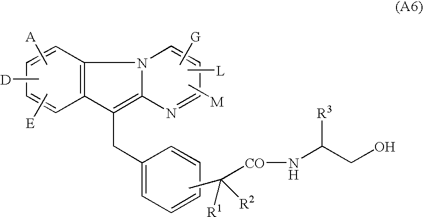
in which
A, D, E, G, L and M are identical or different and are hydrogen, halogen, trifluoromethyl, carboxyl, hydroxyl, straight-chain or branched alkoxy or alkoxycarbonyl each having up to 6 carbon atoms or straight-chain or branched alkyl which has up to 6 carbon atoms and which in turn may be substituted by hydroxyl or by straight-chain or branched alkoxy having up to 4 carbon atoms,
R¹ and R² are identical or different and are hydrogen, cycloalkyl having 3 to 8 carbon atoms or straight-chain or branched alkyl which has up to 10 carbon atoms and which is optionally substituted by cycloalkyl having 3 to 6 carbon atoms, or are phenyl which is optionally substituted by halogen or trifluoromethyl, or
R¹ and R² form, together with the carbon atom, a 4-8-membered cycloalkyl ring,
and
R³ is phenyl which optionally has up to 3 identical or different nitro, carboxyl, halogen, cyano substituents or straight-chain or branched alkenyl or alkoxycarbonyl substituents each having up to 6 carbon atoms, or straight-chain or branched alkyl substituents which have up to 6 carbon atoms and which are optionally substituted by hydroxyl, carboxyl or by straight-chain or branched alkoxy or alkoxycarbonyl each having up to 6 carbon atoms, and/or is optionally substituted by a group of the formula —OR⁴ or —NR⁵R⁶,
in which
R⁴ is hydrogen or straight-chain or branched alkyl or alkenyl each having up to 6 carbon atoms,
R⁵ and R⁶ are identical or different and are phenyl, hydrogen or straight-chain or branched alkyl having up to 6 carbon atoms, or are straight-chain or branched acyl which has up to 8 carbon atoms and which is optionally substituted by a group of the formula —NR⁷R⁸,
in which
R⁷ and R⁸ are identical or different and are hydrogen or straight-chain or branched acyl having up to 8 carbon atoms,
where appropriate in an isomeric form and the salts thereof.

5. The use as claimed in any of claims 1 to 4, characterized in that a compound of Examples 1-119 is employed as MTP inhibitor.

6. The use as claimed in any of claims 1 to 4, characterized in that a compound of Examples 92-119 is employed as MTP inhibitor.

7. The use as claimed in claim 5, characterized in that Example 48 or 80 is employed as MTP inhibitor.

8. The use as claimed in any of claims 1 to 7 for producing medicaments for the prophylaxis and/or control of cardiovascular disorders.

9. The use as claimed in any of claims 1 to 7 for producing medicaments for the prophylaxis and/or control of manifestations of neurodegenerative deficits.

10. The use as claimed in any of claims 1 to 7 for producing medicaments for the therapy of insulin resistance, IGT, diabetes, especially type II diabetes.

11. The use as claimed in any of claims 1 to 7 for producing medicaments for the prophylaxis and/or control of metabolic syndrome.

12. The use as claimed in any of claims 1 to 7 for producing medicaments for the prophylaxis and/or control of obesity.
